(19) United States
(12) Patent Application Publication
(10) Pub. No.: US 2025/0204821 A1
(43) Pub. Date: Jun. 26, 2025

(54) SYSTEMS, DEVICES, AND METHODS FOR DETERMINING AN OXIMETRY VALUE USING AN OXIMETRY MODEL

(71) Applicant: Raydiant Oximetry, Inc., San Ramon, CA (US)

(72) Inventors: Paul Stetson, Piedmont, CA (US); Neil Padharia Ray, Sacramento, CA (US); Adam Jacobs, Hollis, NH (US); Andrew Prescott, Bedford, NH (US); Jana M KAINERSTORFER, Pittsburg, PA (US)

(21) Appl. No.: 18/849,955

(22) PCT Filed: Mar. 24, 2023
(86) PCT No.: PCT/US2023/016309

§ 371 Date: Sep. 23, 2024

Related U.S. Application Data
(60) Provisional application No. 63/323,480, filed on Mar. 24, 2022.

(51) Int. Cl. A61B 5/1464; A61B 5/00; A61B 5/1455; A61B 5/1495; G06T 7/00

(52) U.S. Cl. CPC A61B 5/1464; A61B 5/0022; A61B 5/14552; A61B 5/1495; A61B 5/7267; A61B 5/7282; A61B 5/742; G06T 7/0012; G06T 2207/10048; G06T 2207/30044

(57) ABSTRACT

One or more properties of a pregnant mammal and/or fetus may be used to generate a personalized calibration equation, curve, or formula that may be used to process light transmission data received from a near infrared spectroscopy system used to investigate the pregnant mammal's abdomen. Light detected by a photodetector of and/or associated with the near infrared spectroscopy system may be analyzed using the personalized calibration curve to determine an oxygenation level of the fetus and/or determine a probability of whether, or not, the fetus may be in distress.

This patent application is an INTERNATIONAL PATENT APPLICATION claiming priority to U.S. Provisional Patent Application No. 63/323,480 filed on 24 Mar. 2022 and entitled “SYSTEMS, DEVICES, AND METHODS FOR DETERMINING A FETAL OXIMETRY VALUE USING A MACHINE-LEARNING GENERATED FETAL OXIMETRY MODEL,” which is incorporated by reference herein in its entirety.

### FIELD OF INVENTION

The present invention is in the field of medical devices, oximetry, pulse oximetry, and machine learning. In particular, the present invention is directed to systems, devices, and methods for determining an oximetry value, predicting a hemoglobin, oxygenation status of a subject, and/or detecting, determining, and/or predicting fetal distress using and oximetry model.

### BACKGROUND

Oximetry is a method for determining the oxygen saturation of hemoglobin in a mammal's blood. Typically, 90% (or higher) of an adult human's hemoglobin is saturated with (i.e., bound to) oxygen while only 30-60% of a fetus's blood is saturated with oxygen. Pulse oximetry is a type of oximetry that uses changes in blood volume through a heartbeat cycle to internally calibrate hemoglobin oxygen saturation measurements of the arterial blood.

Current methods of monitoring fetal health, such as monitoring fetal heart rate, are inefficient at determining levels of fetal distress and, at times, provide false positive results indicating fetal distress that may result in the unnecessary performance of a Cesarean delivery.

### SUMMARY

Additionally, or alternatively, described herein are systems, devices, and methods for using simulated light transmission data and associated simulated fetal oximetry values (e.g., fetal hemoglobin oxygen saturation levels and/or fetal tissue oxygen saturation levels) to train a simulated fetal oximetry model. The training may be accomplished using, for example, machine learning, artificial intelligence, a neural network, an artificial neural network, a Bayesian network, and/or deep learning (a portion, or all, of which may be collectively referred to herein as “machine learning”). In some cases, a simulated and/or in vivo fetal oximetry model (as will be discussed below) may be include a plurality of layers and/or functions including, but not limited to, input layers, output layers, confounding factor layers, calculation layers, noise reduction layers, filtering layers, layers regarding an isolation of a fetal portion of light transmission data (e.g., light transmission data that may represent a pulsatile signal of only the fetus) from composite light transmission data that may represent a pulsatile signal of both the pregnant mammal and the fetus, calibration layers, maternal characteristic layers, and/or fetal characteristic layers. In some instances, a simulated and/or in vivo fetal oximetry model may be developed using convolution.

The simulated light transmission data may be generated via the running simulations of plurality of optical inputs through a model of animal tissue (also referred to herein as a “tissue model”). In some embodiments, the simulated light transmission data may be a simulated electronic signal similar to a detected signal generated by a photodetector upon detection of an optical signal (e.g., photons) that may have been incident upon the tissue being modeled (e.g., a pregnant mammal's abdomen and fetus) and then conversion of the detected optical signal into a digital signal. Stated differently, simulated light transmission data may correspond to a simulated electronic signal that is similar to an electronic signal that may be provided by a photodetector upon detection of an optical signal that has traveled through tissue (like the modeled tissue) and conversion of the detected optical signal into a corresponding electronic signal. Often times, the tissue model has at least two layers of tissue and/or blood that may be circulating through tissue that may have different optical properties and, in some instances, one of the layers models/corresponds to maternal tissue (e.g., maternal blood, skin, abdominal wall, uterus, and/or a combination of one or more tissue layers) and another of the layers models/corresponds to fetal tissue (e.g., fetal blood, skin, bone, or neural tissue, and/or a combination thereof). The simulated fetal oximetry model may then be used as a basis to train an in vivo fetal oximetry model using measured in vivo light transmission data and fetal oximetry values via, for example, a process of transfer learning.

This two-step process is beneficial because generating and/or obtaining measured in vivo data sufficient to train a fetal oximetry model from scratch is very difficult given, for example, the number of data points that must be measured and the complexity/cost of obtaining the measured data points. In order to train a fetal oximetry model using only measured in vivo data, a sufficient number (e.g., 5,000-10,000,000) of measured oximetry values in a healthy state (e.g., fetal oxygenation levels are sufficient) and a disease state (e.g., fetal hypoxia and/or fetal hypoxemia) and corresponding light transmission data must be measured and input into the machine learning/model training architecture to train a fetal oximetry model that outputs sufficiently accurate predictions of fetal oximetry values using light transmission data measured in a clinical setting. Currently, measuring fetal oximetry values requires either analysis of a fetal scalp sample taken in-utero, a blood gas analysis conducted on umbilical cord blood following birth or in-utero, and/or a fetal oximetry measurement obtained via an oximeter placed directly on the fetal skin (e.g., cheek or head) via inserting the oximeter into the pregnant mammal's endocervical canal so that it may directly contact the fetal skin. The difficulty of obtaining fetal oximetry measurements along with the relative rarity of fetuses in a disease state provides substantial, even insurmountable, obstacles to obtaining sufficient measured in vivo data to train a fetal oximetry model to predict a fetal oximetry value when given measured light transmission data.

The presently disclosed method solves this problem by using simulated light transmission data and corresponding simulated fetal oximetry values to supply the data needed to train a simulated fetal oximetry model without the need to collect in vivo measure data. This greatly shortens the timeline needed to generate the simulated fetal oximetry model because simulated light transmission data and corresponding simulated fetal oximetry values may be virtually generated relatively quickly using tissue models and simulations without the need to perform invasive and costly medical procedures on a fetus. In addition, the use of simulated light transmission data and corresponding simulated fetal oximetry values allows for the modeling of a variety of scenarios that may occur so rarely clinically that it may take many years to capture sufficient data from these scenarios with which to train a fetal oximetry model solely using measured in vivo data.

By using simulated light transmission data and corresponding simulated fetal oximetry values to train a simulated fetal oximetry model, adapting the simulated fetal oximetry model so that it may be trained using measured in vivo data, and then training an in vivo fetal oximetry model using measured in vivo data, the a timeline for process of generating a valid and clinically useful fetal oximetry model is greatly shorted and is more accurate because a portion (e.g., 40-95%) of the training of the in vivo fetal oximetry model is already completed via the training of the simulated fetal oximetry model without the need for costly and difficult to obtain measured in vivo data.

The methods disclosed herein may be executed by processors, or networks of processors, that are configured to perform machine learning and/or deep machine learning processes to develop predictive models, in this case models that can receive light transmission data that includes light that was incident on a fetus, analyze the light transmission data, and predict a fetal oximetry value with sufficient precision to be clinically useful when, for example, determining whether a fetus is in distress during, for example, gestation and/or a labor and delivery process. In some cases, the processors, or networks of processors may reside in a cloud computing environment. In some embodiments, the processor is and/or includes a machine learning architecture.

In some embodiments, a plurality of sets of simulated light transmission data and corresponding oximetry values for each set of simulated light transmission data may be received. At times, a fetal oximetry value for each set of simulated light transmission data may be calculated using the respective set of simulated light transmission data via, for example, the Beer-Lambert Law or modified Beer-Lambert Law. Each set of the simulated light transmission data may have been generated by simulating a transmission of light through a model of animal tissue, wherein the model includes at least two layers of animal tissue with one of the layers of the model of animal tissue modeling fetal tissue. In some embodiments, each layer of the animal tissue model may have different optical properties (e.g., absorption, scattering, etc.).

In some instances, the plurality of sets of simulated light transmission data may include simulated light transmission data for light of one or more wavelengths or distinct ranges of wavelengths such as light with a wavelength within a range of 620 nm-670 nm, 920 nm-970 nm, 640 nm-660 nm, or 940-960 nm. Additionally, or alternatively, the simulated light may be of a broadband (e.g., white light) of wavelengths.

A simulated fetal oximetry model may then be trained using the plurality of sets of simulated light transmission data and corresponding oximetry values by, for example, inputting the plurality of sets of simulated light transmission data and corresponding oximetry values into a machine learning architecture. The simulated fetal oximetry model may include a plurality of layers and/or functions and may be configured to receive light transmission data and determine an oximetry value for a fetus using the received light transmission data.

Instructions to adapt the simulated fetal oximetry model for transfer to an in vivo fetal oximetry model may be received and, once the simulated fetal oximetry model is sufficiently trained using the sets of simulated light transmission data, the simulated fetal oximetry model may be adapted for transfer to an in vivo fetal oximetry model responsively to the instructions.

In some embodiments, an instruction to adapt the simulated fetal oximetry model for transfer to an in vivo fetal oximetry model may be received. These instructions may include instructions to fix, or lock, one or more layers of the simulated fetal oximetry model (e.g., an input layer, a calibration layer, a maternal characteristic layer, a fetal characteristic layer, a noise cancelling layer, etc.) that are generally applicable to the in vivo fetal oximetry model so that the fixed layers do not change during the training process for the in vivo fetal oximetry model. Exemplary inputs to the one or more fixed layers of the simulated fetal oximetry model may correspond to a calibration factor for determining an oximetry value, a calibration curve for determining an oximetry value, a calibration formula for determining an oximetry value, a calibration model for determining an oximetry value, a wavelength, or a range of wavelengths, of light in the simulated light transmission data from a given distance between a source and a detector, a fetal depth and/or a physiological and/or geometrical characteristic of the pregnant mammal and/or fetus

A plurality of sets of measured in vivo light transmission data corresponding light traveling through and being emitted from (e.g., via backscattering) an abdomen of a pregnant mammal and her fetus may then be received. Each set of measured in vivo light transmission data may correspond to a fetal oximetry value, which may also be received. Then, an in vivo fetal oximetry model may be generated and/or trained by inputting the plurality of sets of measured in vivo light transmission data and corresponding measured fetal oximetry values into the adapted simulated fetal oximetry model. Once training of the in vivo fetal oximetry model is complete, it may be stored in a database and/or an indication that the training of the in vivo fetal oximetry model is complete may be provided to a user via, for example, a display device.

In some instances, the plurality of sets of measured, in vivo light transmission data may include light transmission data for light of one or more wavelengths or distinct ranges of wavelengths such as light with a wavelength within a range of 620 nm-670 nm, 920 nm-970 nm, 640 nm-660 nm, or 940-960 nm. Additionally, or alternatively, the simulated light may be of a broadband (e.g., white light) of wavelengths.

In some embodiments, the fetal oximetry values may be fetal hemoglobin oxygen saturation values and a set of measured light transmission data for a pregnant mammal may be received. The light may have been incident on the pregnant mammal's abdomen and a fetus positioned within the pregnant mammal's abdomen. A fetal hemoglobin oxygen saturation value may be determined for the fetus' blood by inputting the set of measured light transmission data into the in vivo fetal oximetry model. The fetal hemoglobin oxygen saturation value for the fetus' blood may then be communicated to a display device.

Additionally, or alternatively, the fetal oximetry values may be fetal tissue oxygen saturation values and a set of measured light transmission data for a pregnant mammal incident on the pregnant mammal's abdomen and a fetus positioned within the pregnant mammal's abdomen. A fetal tissue oxygen saturation value for a portion of fetal tissue may them be determined by inputting the set of measured light transmission data into the in vivo fetal oximetry model. The fetal tissue oxygen saturation value for the portion of fetal tissue may then be communicated to a display device.

Additionally, or alternatively, an additional plurality of sets of measured in vivo light transmission data for light traveling through an abdomen of the pregnant mammal may be received. At least some of the measured in vivo light transmission data may correspond to light incident on the fetus and, at times, a portion of the light transmission data corresponding to light that is isolated from light incident only on the pregnant mammal so that a pulsatile signal of the fetus and/or tissue of the fetus may be isolated from the light transmission data. The in vivo fetal oximetry model may then be updated by inputting the additional plurality of sets of measured in vivo light transmission data and corresponding measured fetal oximetry values into the in vivo fetal oximetry model, thereby generating an updated in vivo fetal oximetry model. The updated in vivo fetal oximetry model may be stored in a database and/or used to predict a fetal oximetry value using in vivo light transmission data measured in, for example, a clinical setting.

In some cases, the training of the simulated fetal oximetry model may include using machine learning to train the simulated fetal oximetry model. Additionally, or alternatively, the training of the in vivo fetal oximetry model may include using machine learning to train the in vivo fetal oximetry model.

At times, the in vivo fetal oximetry model may be configured to receive measured in vivo light transmission data and predict fetal hypoxia and/or fetal hypoxemia using the received measured in vivo light transmission data. Additionally, or alternatively, the wherein in vivo fetal oximetry model may be configured to receive measured in vivo light transmission data and predict a fetal oximetry value using the received measured in vivo light transmission data.

In some embodiments, a fetal oximetry value predicted by the in vivo fetal oximetry model may be compared to a threshold fetal oximetry value and an indication of the comparison to a display device. At times, the indication is an alert when, for example, the fetal oximetry value is below the threshold fetal oximetry value. In some instances, the set of measured light transmission data may be a first set of measured light transmission data and the determined fetal oximetry value may be a first determined oximetry value and a second set of measured light transmission data for a pregnant mammal may be received. A second fetal oximetry value may then be determined for the fetus by inputting the second set of measured light transmission data into the in vivo fetal oximetry model. A relationship (e.g., a trend) between the first and second fetal oximetry values may be determined and then an indication of the relationship to a display device.

In some embodiments, systems, devices, and methods may be configured so that light transmission data corresponding to an optical signal that is detected by a photodetector and converted into the light transmission data is received by a processor. The optical signal may be a composite of light that is incident on a pregnant mammal's abdomen and a fetus contained within the pregnant mammal's abdomen. The light transmission data may be input into an in vivo fetal oximetry model that has been trained using simulated light transmission data. An output from the in vivo fetal oximetry model, the output including an indication of an oximetry value for the fetus. The oximetry value may be, for example, a level of fetal hemoglobin oxygen saturation, and/or a level of fetal tissue oxygen saturation.

At times, the systems, devices, and/or methods disclosed herein may be configured to isolate a portion of the light transmission data that corresponds to light that was incident on the fetus and thereby isolate a fetal signal prior to inputting the light transmission data into the in vivo fetal oximetry model, wherein the fetal signal is input into the in vivo fetal oximetry model.

Additionally, or alternatively, in some embodiments, the in vivo fetal oximetry model may be iteratively tuned, over time and clinical usage with additional measured in vivo light transmission data.

In some embodiments, the systems, devices, and/or methods disclosed herein may be configured to provide an indication of the oximetry value for the fetus to a display device and/or store an indication of the oximetry value for the fetus in a database.

In some embodiments, the systems, devices, and/or methods disclosed herein may be configured to determine whether the fetus has fetal hypoxia and/or fetal hypoxemia using the fetal oximetry value and an indication of this determination may be provided to a display device.

Additionally, or alternatively, the systems, devices, and/or methods disclosed herein may be configured to compare a predicted fetal oximetry value to a threshold fetal oximetry value and provide an indication of the comparison to a display device. In some instances, the indication is an alert when the fetal oximetry value is below the threshold fetal oximetry value.

Exemplary devices disclosed herein include 1) a communication interface configured to communicate with a display device and a source of light transmission data to receive a set of light transmission data; 2) a memory having an in vivo fetal oximetry model stored thereon; and 3) a processor configured to receive light transmission data from the communication interface, access the in vivo fetal oximetry model stored in the memory, predict a fetal oximetry value by inputting the received light transmission data into the in vivo fetal oximetry model, and communicate an indication of the fetal oximetry value to the display device.

In some embodiments, the processor may be further configured to isolate a portion of the light transmission data that corresponds to light that was incident on the fetus, thereby generating a fetal signal prior to inputting the light transmission data into the in vivo fetal oximetry model, wherein the fetal signal is input into the in vivo fetal oximetry model. Additionally, or alternatively, the processor may be further configured to store an indication of the fetal oximetry value for the fetus in a database.

Exemplary systems disclosed herein may include a fetal oximetry probe, a memory having an in vivo fetal oximetry model stored thereon, and a processor configured to receive light transmission data from the communication interface, access the in vivo fetal oximetry model stored in the memory, predict a fetal oximetry value by inputting the received light transmission data into the in vivo fetal oximetry model, and communicate an indication of the fetal oximetry value to the display device in accordance with one or more embodiments disclosed herein. The fetal oximetry probe may include, for example, one or more light source(s) configured to shine light into a pregnant mammal's abdomen and a fetus contained therein, one or more detectors (e.g., photodetectors) configured to detect light, from the light source, emanating from the pregnant mammal's abdomen and fetus and convert the detected light into light transmission data, and a communication interface configured to communicate the light transmission data to a processor.

At times, systems, devices, and methods disclosed herein may be configured to receive an image (e.g., ultrasound, CT scan image, and/or MRI image) of a pregnant mammal's abdomen and/or a fetus contained with the pregnant mammal's abdomen. The image may be analyzed to determine one or more characteristics of the pregnant mammal, the pregnant mammal's abdomen, and/or the fetus. The characteristics may be, for example, an optical characteristic, a geometrical characteristic, and a physiological characteristic.

Light transmission data corresponding to an optical signal incident on the pregnant mammal's abdomen, and, in some cases, the pregnant mammal's fetus may be received. The light transmission data may have been detected by a photodetector and converted into the light transmission data as, for example, a digital and/or analog signal. The light transmission data may then be input into an in vivo fetal oximetry model that has been personalized to the pregnant mammal and/or fetus responsively to a result of the analysis of the image and an output from the personalized in vivo fetal oximetry model may then be received. The output may be communicated to a display device (e.g., screen) for communication to a user and/or clinician. The output may include an indication of an oximetry value for the fetus such as a level of fetal hemoglobin oxygen saturation and/or a level of fetal tissue oxygen saturation. In some embodiments, the output and/or oximetry value for the fetus may be analyzed and/or processed to determine whether or not the fetus may be in distress, has fetal hypoxia or fetal hypoxemia. An indication of whether or not the fetus may be in distress, has fetal hypoxia or fetal hypoxemia may be provided to the display device.

In some embodiments, a result of the analysis of the image may be to calculate, generate, and/or determine a calibration equation for the pregnant mammal that may be incorporated into an in vivo fetal oximetry model, thereby personalizing the in vivo fetal oximetry model. The calibration equation may, for example, calibrate received light transmission data, analysis of the received light transmission data, and/or results of an analysis and/or processing of the received light transmission data to calibrate for maternal and/or fetal optical, geometrical, and/or physiological characteristics.

Additionally, or alternatively, an in vivo fetal oximetry model may be personalized using a characteristic of equipment used to generate the optical signal, a characteristic of the photodetector, a light scattering coefficient specific to the pregnant mammal and/or fetus, a light absorption coefficient specific to the pregnant mammal and/or fetus, a skin color of the pregnant mammal's abdomen, and/or a blood oxygen level of the pregnant mammal.

Additionally, or alternatively, a database of known, or pre-calculated, calibration equations may be queried for one or more calibration equations that may be responsive to the analysis of the image and/or one or more characteristics of the pregnant mammal and/or fetus (that may, or may not, be determined responsively to the analysis of the image). The queried for calibration equation(s) may be received from the database and incorporated into the personalized in vivo fetal oximetry model and/or incorporated into an in vivo fetal oximetry model, thereby generating a personalized the in vivo fetal oximetry model.

In another embodiment, information regarding a blood oxygen value of a fetus may be determined by obtaining one or more signal(s) indicating and/or corresponding to light detected from a pregnant mammal's abdomen and/or a fetus disposed in the pregnant mammal's abdomen following application of and optical signal (e.g., light) to the pregnant mammal's abdomen.

The one or more signal(s) may be analyzed and/or processed using one trained model(s) and/or calibration formulas that, in some cases, may be personalized and/or tuned to the pregnant mammal, her fetus, and/or a characteristic of the pregnant mammal, the pregnant mammal's abdomen, and/or the fetus disposed within the pregnant mammal's abdomen. A result of the processing and/or analysis performed by the one or more trained models may be to determine blood and/or tissue oxygen information for the pregnant mammal and/or fetus that may be used to determine the information regarding a blood and/or tissue oxygen value of the fetus. Additionally, or alternatively, a result of the processing and/or analysis performed by the one or more trained models may be to determine whether the fetus is in distress (e.g., fetal hypoxia or fetal hypoxemia) and, if so, a degree of that distress.

At times, one or more of the trained model(s) may be personalized to the pregnant mammal and/or fetus using one or more characteristics of the pregnant mammal, the pregnant mammal's abdomen, and/or the fetus. On some occasions, the characteristic may be determined by analyzing one or more images of the pregnant mammal, the pregnant mammal's abdomen, and/or the fetus. Additionally, or alternatively, the characteristic may be determined by taking one or more measurements and/or observations of the pregnant mammal, the pregnant mammal's abdomen, and/or the fetus. Exemplary characteristics that may be used to personalize one or more trained models and/or select one or more trained models from a plurality of trained models that may be stored in, for example, a database disclosed herein are optical (e.g., light scattering coefficient of/specific to the pregnant mammal, a light absorption coefficient of/specific to the pregnant mammal, and/or maternal skin tone), geometric (e.g., thickness, orientation), and physiological (e.g., oximetry values, uterine tone, tissue type, and/or a blood oxygen level of the pregnant mammal, etc.) characteristics of the pregnant mammal, the pregnant mammal's abdomen, and/or the fetus. Additionally, or alternatively, the trained model may be personalized to the pregnant mammal using, for example, a characteristic of the at least one signal, a characteristic of a light source projecting light in the pregnant mammal's abdomen that corresponds to the at least one signal, a characteristic of a photodetector that detects the light from the pregnant mammal's abdomen that corresponds to the at least one signal.

In some embodiments, the trained model may be personalized to the pregnant mammal responsively to analysis of an image of the pregnant mammal's abdomen and/or fetus. Additionally, or alternatively, the trained model may be personalized to the pregnant mammal using a calibration formula that may be responsive to one or more characteristics of the pregnant mammal as determined from, for example, analysis of the image and/or a geometrical and/or physiological measurement.

In one or more alternate, or additional, embodiments, information regarding a blood oxygen value of a patient may be determined by, for example, obtaining one or more signal(s) associated with and/or indicative of light detected from the patient following application of light to the patient.

The signal(s) may then be analyzed using one or more trained model(s) that, in some cases, may be personalized to the patient, to determine blood oxygen information for the patient such as a blood hemoglobin oxygenation level or percentage and/or a tissue oxygenation level. The trained model may be personalized to the patient using at least one of an optical, geometric, and physiological characteristics of the patient.

In some embodiments disclosed herein, one or more trained models, simulated fetal oximetry models, in vivo fetal oximetry models, calibration equations and/or correction factors may be used in combination to, for example, determine oximetry values for a patient, a fetus, and/or a pregnant mammal.

In some embodiments, the systems, devices, and methods disclosed herein may be configured to generate one or more calibration equations that are specific and/or personalized to a pregnant mammal, the pregnant mammal's fetus, and/or a combination of the pregnant mammal's abdomen and the fetus disposed therein. The personalized calibration equations may be generated using, for example, optical, geometrical, and/or physiological properties of the pregnant mammal and/or fetus. For example, in some embodiments, one or more optical properties of a pregnant mammal and/or a pregnant mammal's abdomen may be received and/or determined via, for example, analysis of an image of the pregnant mammal's abdomen, analysis of a short separation measurement that investigates one or more maternal layers of tissue with light, a scattering coefficient for the pregnant mammal's abdomen, an absorption coefficient for the pregnant mammal's abdomen, and/or a skin tone for the pregnant mammal's abdomen. One or more of these optical properties may then be used to generate and/or adjust a calibration equation that is specific or personalized to the pregnant mammal. The calibration equation may be used to, for example, analyze light that has traveled through the pregnant mammal's abdomen and is detected by a photodetector so that, for example, an oximetry value for the pregnant mammal and/or fetus may be determined.

Additionally, or alternatively, in some embodiments, a physiological property of the pregnant mammal's abdomen and a second calibration equation for the pregnant mammal may be generated using/responsively to the received physiological property. Additionally, or alternatively, a physiological property of a fetus disposed in pregnant mammal's abdomen may be received and a calibration equation for the fetus and/or combination of the fetus and pregnant mammal's abdomen (e.g., a third calibration equation) may be generated responsively to the received physiological property of the fetus. Additionally, or alternatively, an optical property of a fetus disposed in pregnant mammal's abdomen may be received and a calibration equation for the fetus and/or combination of the fetus and pregnant mammal's abdomen (e.g., a fourth calibration equation) may be generated responsively to the received optical property of the fetus.

In these embodiments, light transmission data corresponding to an optical signal that is incident on the pregnant mammal's abdomen and the fetus disposed within the pregnant mammal's abdomen and is detected by a photodetector and converted into the light transmission data. Then, an oximetry value for the fetus by applying at least one or more of the first calibration equation, the second calibration equation, the third calibration equation, and the fourth calibration equation to the light transmission data.

Additionally, or alternatively, in some embodiments, an image and/or characteristic of a pregnant mammal's abdomen may be received and analyzed to determine one or more characteristics of the pregnant mammal's abdomen. The image may show, for example, one or more layers of maternal tissue, the fetus, and/or characteristics thereof. Then, optical data regarding the pregnant mammal's abdomen and/or physiological data for the pregnant mammal may be received and used to a calibration equation for the pregnant mammal, which may be referred to herein as a “personalized calibration equation.” The physiological and/or optical data for the pregnant mammal may be received from, for example, a frequency domain near infrared spectroscopy system, a time of flight near infrared spectroscopy system, and/or a continuous wave near infrared spectroscopy system. At times, the physiological data for the pregnant mammal includes an oxygen saturation level for the pregnant mammal that may be received from a pulse oximeter.

At times, light transmission data may be received and an oximetry value for the fetus may be determined by applying and/or processing the personalized calibration equation to/with the light transmission data.

In some embodiments, receiving optical data and/or physiological data for the fetus may be received and the calibration equation may be further responsive to the received optical and/or physiological data for the fetus. In some cases, the physiological and/or optical data for the fetus may be received from a time of flight near infrared spectroscopy system, a continuous wave near infrared spectroscopy system, and/or a time of flight near infrared spectroscopy system. Additionally, or alternatively, the light transmission data may be received from a frequency domain near infrared spectroscopy system, a continuous wave near infrared spectroscopy system, and/or a time of flight near infrared spectroscopy system.

In some instances, a plurality of sets of light transmission data may be received from one or more photodetectors and/or spectroscopy systems. For example, a first set of light transmission data may be received from a first spectroscopy system and a second set of light transmission data may be received from a second spectroscopy system. The plurality of sets transmission data may then be processed by applying the calibration equation thereto when determining an oximetry value for the fetus.

### BRIEF DESCRIPTION OF THE FIGURES

The present invention is illustrated by way of example, and not limitation, in the figures of the accompanying drawings in which:

FIG. 1A is a block diagram illustrating an exemplary system for developing a model to accurately calculate fetal oxygen saturation in-utero, consistent with some embodiments of the present invention;

FIG. 1B is a block diagram of an exemplary system for detecting and/or determining fetal hemoglobin oxygen saturation levels, consistent with some embodiments of the present invention;

FIG. 1C is a block diagram of an exemplary fetal oximetry probe that may be used in the system of FIG. 1B, consistent with some embodiments of the present invention;

FIG. 2A is a flowchart showing an exemplary process for generating a plurality of sets of simulated light transmission data and corresponding oximetry values using a computer-generated model of animal tissue, consistent with some embodiments of the present invention;

FIG. 2B depicts a graph plotting exemplary relationships between a ratio of ratios (R) and a hemoglobin oxygen saturation percentage of a fetus for various fetal depths, consistent with some embodiments of the present invention;

FIG. 2C depicts a graph that plots exemplary relationships between a ratio of a change in an absorption coefficient for an infrared wavelength of light divided by a ratio of a change in an absorption coefficient for a red wavelength of light is related to a hemoglobin oxygen saturation percentage of a fetus for various fetal depths, consistent with some embodiments of the present invention;

FIG. 3 is a flowchart showing an exemplary process for generating a plurality of sets of simulated light transmission data and corresponding oximetry values using light transmitted through a physical model of animal tissue, consistent with some embodiments of the present invention;

FIG. 4A is a flowchart illustrating a first part of an exemplary process for developing a model to compensate for the physio-optical influences of transabdominal fetal oximetry in order to accurately calculate fetal oxygen saturation in-utero, in accordance with some embodiments of the present invention;

FIG. 4B is a flowchart illustrating a second part of the exemplary process of FIG. 4A, in accordance with some embodiments of the present invention;

FIG. 5 is a flowchart illustrating a process for the generation of a tuned simulated fetal oximetry model, consistent with some embodiments of the present invention;

FIG. 6 is a flowchart illustrating a process for the generation of a tuned in vivo fetal oximetry model, consistent with some embodiments of the present invention;

FIG. 7 is a flowchart illustrating an exemplary process for the generation of an in vivo fetal oximetry model, consistent with some embodiments of the present invention;

FIG. 8 is a flowchart illustrating a process for the determination of an oximetry value for a fetus using an in vivo fetal oximetry model, consistent with some embodiments of the present invention;

FIG. 9 is a diagram showing an exemplary seven-layer two-dimensional model of a pregnant mammal's abdomen and fetus is shown, in accordance with some embodiments of the present invention;

FIG. 10 is a table of an exemplary set of parameters, in accordance with some embodiments of the present invention;

FIG. 11 provides a graph that plots a simulated fetal and maternal photoplethysmogram (PPG) over time in seconds, in accordance with some embodiments of the present invention;

FIG. 12A provides a flowchart of an exemplary process for using an in vivo fetal oximetry model to determine a fetal oxygenation value, in accordance with some embodiments of the present invention;

FIG. 12B is an image of a pregnant woman's abdomen taken using magnetic resonance imaging;

FIG. 12C is a rendering of the image of FIG. 12B following processing via execution of one or more processes disclosed herein, in accordance with some embodiments of the present invention;

FIG. 12D is a detailed view of a portion of the rendering of FIG. 12C, in accordance with some embodiments of the present invention;

FIG. 13 provides a flowchart of an exemplary process for selecting a calibration formula for use with an in vivo fetal oximetry model and determine a fetal oxygenation value using the calibration formula and an in vivo fetal oximetry model, in accordance with some embodiments of the present invention;

FIG. 14 provides a flowchart of an exemplary process for determining optical properties of maternal tissue, selecting a calibration formula for use with an in vivo fetal oximetry model responsively to the maternal optical properties, and determining a fetal oxygenation value using the calibration formula and an in vivo fetal oximetry model, in accordance with some embodiments of the present invention;

FIG. 15 provides a flowchart of another exemplary process for determining optical properties of maternal tissue, selecting a calibration formula for use with an in vivo fetal oximetry model responsively to the maternal optical properties, and determining a fetal oxygenation value using the calibration formula and an in vivo fetal oximetry model, in accordance with some embodiments of the present invention;

FIG. 16 provides a flowchart of an exemplary process for determining a fetal oximetry value of using a calibration formula and an in vivo fetal oximetry model, in accordance with some embodiments of the present invention;

FIG. 17 provides a flowchart of an exemplary process for generating a fetal signal, in accordance with some embodiments of the present invention;

FIG. 18 provides a flowchart of an exemplary process for generating a fetal signal, in accordance with some embodiments of the present invention; and

FIG. 19 is a screen shot of an exemplary user interface configured to display an oximetry value and/or indication of fetal distress, in accordance with some embodiments of the present invention.

Throughout the drawings, the same reference numerals, and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### Written Description

Near-IR spectroscopy, and pulse oximetry calculations, to estimate a percentage of oxygen bound to hemoglobin in the blood (also referred to herein as “hemoglobin oxygen saturation” or “SpO2”) may incorporate calculations using the modified Beer Lambert law (mBLL), which describes changes in hemoglobin absorption related to changes in light intensity of various wavelengths. Under the assumption that bulk tissue has homogenous characteristics, the modified Beer Lambert law can be written as Equation 1:

$$Δ {μ}_{a} ( λ ) = - \frac{1}{r * D PF ( λ )} \frac{{I}_{d ( λ )} - {I}_{s ( λ )}}{{I}_{s ( λ )}} = - \frac{1}{r * D PF ( λ )} Δ O D \qquad Equation 1$$

Where:
   I_{d}=diastolic intensity of the fetal pulse
   Iₛ=systolic intensity of the fetal pulse
   r=source detector distance, which may be given by the geometry of an optical probe or source/detector combination
   DPF=differential path length factor, which is not known
   ΔOD=change in optical density
   Δμₐ=change in absorption coefficient
   λ=wavelength

Results from this equation may be used to extract values for concentrations of oxygenated hemoglobin (sometimes referred to herein as “c_HbO”) and deoxygenated hemoglobin (sometimes referred to herein as “c_Hb”) 2 according to Equation 2, below.

$$SpO 2 ( \% ) = c\_HbO / ( c\_HbO + c\_Hb ) \qquad Equation 2$$

Although the modified Beer Lambert Law traditionally serves as the fundamental basis for near-IR spectroscopy, it is limited by several assumptions, including that light absorption within tissue is homogeneous, change in a differential path length factor is negligible, and that the light scattering within tissue is low. In complex in vivo, physio-optical environments of, for example, non-homogenous tissue and/or two or more layers of different types of tissue such as a fetus within a mother, these assumptions may not always hold true and yield accurate calculations. For example, traditional methods for calculating pulse oximetry using the modified Beer Lambert Law assume that photons travel a relatively short distance (e.g., 1 cm) and that there is a negligible change in the differential path length factor (DPF) across this distance. However, when a distance photons travel is larger than 1 cm (e.g., 1.5-10 cm) changes in the DPF can be significant thereby adversely impacting the accuracy SpO2 calculations. For example, in transabdominal fetal application, photons can travel 5 cm, 10 cm, or more and, consequently, changes in the DPF can be significant. To accurately account for DPF variability and calculate oxygen saturation is a complex problem, particularly when multiple wavelengths of light are used. One way to overcome this problem is to calculate SpO2 using a 2-layer description of the modified Beer Lambert Law that can independently calibrate for different layers of tissue.

However, a drawback of the using the two-layer Modified Beer Lambert Law calculations when calculating oxygen saturation of target tissue is that this approach is dependent on having an accurate depth of the target (e.g., a fetus) within the body input in order to accurately calculate the blood oxygen saturation. This may pose a challenge in a clinical situation because measuring (via, for example, an ultrasound or Doppler device) depth of a target (e.g., a fetus, fetal head, or fetal back) within a body is open to clinical interpretation and may not always be reliable, especially when differences of as little as 5 mm can impact the accuracy of calculations. Furthermore, a target's depth that is measured by an ultrasound or Doppler device may not accurately reflect that path of the photons because, for example, the photons are a different signal type (i.e., optical) than the sound waves used by the ultrasound/Doppler device and/or the ultrasound/Doppler device is not positioned where the optical probe is positioned so the ultrasound/Doppler device may be imaging a portion of the surface tissue that is not coincident with the placement of the optical probe. In order to overcome the target depth requirement, machine learning may be deployed as a methodology to develop a model that augments the 2-layered description of the modified Beer Lambert Law to arrive at target SpO2 values without requiring target depth as an input. This model would them be able to accurately determine fetal oximetry values without requiring fetal depth.

Transdermal in vivo measurements of target (e.g., fetal) SpO2 levels may involve placing an optical probe (e.g., one or more light source(s) and photodetector(s)) on the skin of a patient (e.g., a pregnant woman), transmitting an optical signal into the skin of the patient, and collecting resulting optical signals emitted from the skin of the patient via, for example, backscattering and/or transmission through the patient's non-target tissue and target's tissue. In many cases, determining SpO2 involves calculating the amplitudes of the AC signals, normalizing them using (dividing by) the amplitude of the DC signals, and multiplying the normalized AC signals by a calibration factor that takes into account, for example, abdominal and/or fetal tissue scattering properties and/or fetal depth as a function of wavelength of the incident optical signal/light. However, this normalization methodology is, in many cases, too generic of an approach because, for example, the impact of the maternal/fetal tissue on the behavior of the incident light is uniform along the pathlength of the optical signal. However, in many cases, this is inaccurate due to one or more confounding influences of, for example, maternal tissue, maternal physiology, maternal movement, and/or fetal tissue/physiology/movement when the optical probe is being used and/or during measurement of fetal SpO2. Hence, a different normalization methodology for the AC signals may assist with more accurately calculating fetal SpO2.

In some embodiments, using one or more optical probe(s) that include multiple light sources (also called “sources” herein) and/or multiple photodetectors (also called “detectors” herein) that facilitate multiple sets of sources and detectors that have different source-detector distances (i.e., different distances between the source and detector) may provide inputs that can be used to compensate for confounding influences in some situations because, for example, a mean depth of penetration for the light into the patient's tissue (e.g., pregnant mammal's abdomen) gets larger as the source-detector separation increases. Shorter separations between the source and detector result in light that penetrates the tissue less deeply than light from larger separations between the source and detector. Use of signals detected when the source/detector distance is relatively short biases these measurement towards measuring only the patient's non-target (e.g., maternal or abdominal) tissues (which are shallower than the target tissue), because the light detected by relatively close detectors only penetrates the patient's non-target tissue. In some cases, these detected signals may be called short separation signals.

Additionally, or alternatively, by using one or more probes with various source/detector distances both the patient's non-target-only signals (i.e., short separation signals) and a composite signal that includes light incident on both the patient's non-target and target tissue (sometimes referred to herein as a “composite signal”) may enable measurement and/or comparisons of variability of the patient's non-target and/or target tissue. In some instances, comparing detected signals from detectors with a short source/detector distance with detected signals from detectors with a longer source/detector distance may facilitate understanding of how the patient's non-target and/or target tissue my impact the behavior of light incident thereon. This information may be used, for example, to normalize AC signals, develop or adjust a calibration formula used to determine target SpO2, and/or develop or adjust a calibration factor used to determine target SpO2, which may make calculation of target SpO2 more accurate than previously used techniques.

In, for example, a transabdominal fetal oximetry context, using machine learning as a methodology to develop a model that augments the 2-layered (one maternal and one fetal) description of the modified Beer Lambert Law to arrive at fetal hemoglobin oxygen saturation values without requiring fetal depth as an input and/or incorporates confounding influences of maternal and fetal tissue when determining fetal SpO2 requires a large data set of fetal SpO2 values so that many different scenarios with different fetal depths and/or confounding factors may be understood and factored into a determination of fetal SpO2 in a particular situation. A data set of fetal oximetry and/or SpO2 values calculated using a model, or mathematical simulation, of the fetal and/or maternal tissue, which is sometimes referred to herein as “calculated fetal oximetry values” or “calculated fetal Spo2 values,” may be used to train and/or test a processor to determine fetal SpO2 values. In some instances, the model may be a physiological model of the fetus and mother, which in some cases may include static and time variant tissue layer properties of the fetus and/or pregnant mammal that may calculate how light may behave when transmitted and/or detected with various source-separation distances and light wavelengths. These calculated SpO2 values may, in some cases, represent a simulated light transmission time series data set (“also referred to herein as a “simulated light transmission data set”) that models the optical signals that may be detected by a detector over time and may thereby be available for analysis, manipulation, and input into machine learning models in a manner similar to, for example, actual light transmission data sets collected from a detector and/or actual fetal SpO2 values. In some embodiments, simulated light transmission data used to calculate fetal oximetry and/or SpO2 values may be determined and/or generated using machine learning equipment and/or techniques.

Additionally, or alternatively, the simulated light transmission data may be generated by software designed to build models and/or generate simulated data for light traveling through tissue and/or tissue model(s). Examples of this software are Monte Carlo simulations and Near Infrared Fluorescence and Spectral Tomography (NIRFAST, Dartmouth College, NH) software. Use of modeling software allows for models to be built that incorporate a variety of parameters such as wavelength of light used, DFP, source/detector distance, and/or maternal and/or fetal morphological, geometric and/or physiological parameters such as abdominal wall thickness and/or composition, tissue composition, tissue type, muscular state of the maternal uterus, maternal skin color, fetal skin color, and/or position on the fetus on which the light was incident. At times, the parameters of the data sets and/or inputs used to generate the models may be changed discretely, randomly, pseudo-randomly, and/or selected within a range and/or distribution of values. Additionally, or alternatively, combinations of input parameters may be used to generate the simulated signals. This approach and/or a combination of approaches may provide a random covering of the possible simulated light transmission data sets/time series and/or calculated fetal SpO2 values that may be used for training and testing the machine learning model. Additionally, or alternatively, features may be extracted from simulated light transmission data sets to be used as inputs to the machine learning architecture or models. Examples of potential features that may be extracted from simulated light transmission data sets are correlation amplitudes, FFTs, time of flight for photons exiting the maternal abdomen, DC levels, AC levels or other post-processed signal descriptors.

Possible uses and/or advantages of the present invention include, but are not limited to, facilitation of perturbation analysis of the data sets whereby one variable (e.g., maternal heart rate, fetal heart rate, fetal distance, source/detector distance) is changed at a time to determine an impact (if at all) on the calculated fetal SpO2 values. This is a substantial advantage over experimentally determined data sets, or calculated fetal SpO2 values, because it is difficult, in real life, to control only one factor at a time because, often times, multiple factors change at unpredictable rates/times with in vivo situations.

Additionally, or alternatively, the present invention may be used to perform sensitivity analysis, which may allow for changing multiple variables/parameters used to generate the models and/or simulated light transmission data sets so that, for example, the results (e.g., calculated fetal SpO2 values) may be evaluated for accuracy and/or to determine how multiple variables may interact with one another to vary calculated fetal SpO2 values. Model variables that may be modified to perform sensitivity analysis include, but are not limited to, noise, wavelength of light used, DFP, source/detector distance, and/or maternal and/or fetal morphological, geometric and/or physiological parameters such as abdominal wall thickness and/or composition, tissue composition, tissue type, muscular state of the maternal uterus, maternal skin color, fetal skin color, and/or position on the fetus on which the light was incident.

Additionally, or alternatively, an advantage of the present invention is that use of simulated light transmission data sets and/or fetal SpO2 values calculated using the simulated light transmission data sets to train a simulate fetal oximetry model, or teach the machine, reduces the number of experimentally, or measured, in vivo light transmission data sets and/or fetal SpO2 values that are necessary to arrive at an accurately trained model. This, in turn, reduces the need for a very large and difficult to obtain data set of actual fetal SpO2 values determined using measured/in vivo data (e.g., a blood gas analysis).

In some instances, the systems, methods, and devices, disclosed herein may be used to assist clinicians and/or users to assess fetal wellbeing and/or determine and/or predict whether a fetus is in distress prior to and/or during a labor and delivery process. At times, the systems, methods, and devices, disclosed herein may be used in addition to traditional fetal monitoring methods and devices (e.g., monitoring fetal heart rate) to achieve higher reliability in assessing fetal health and/or predicting fetal distress than traditionally available methods.

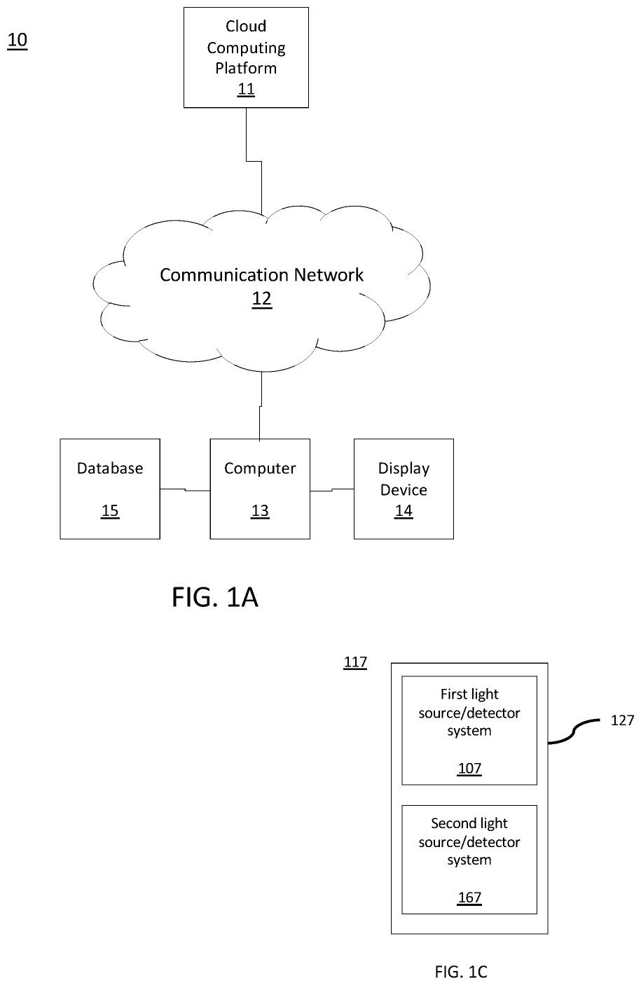

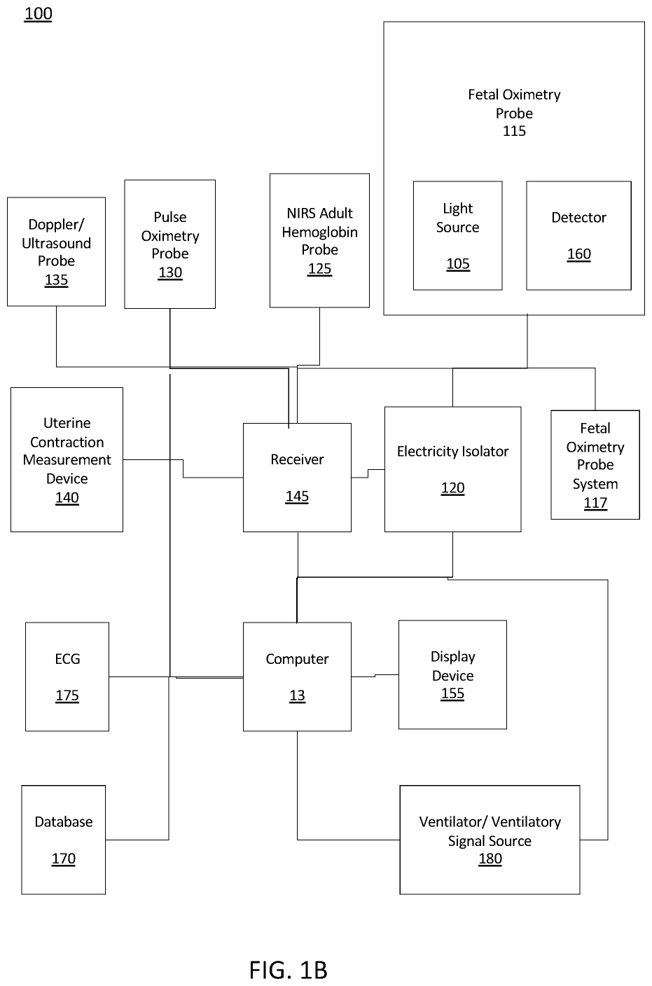

FIG. 1A provides an exemplary system 10 for using machine learning to develop a simulated fetal oximetry model and/or an in vivo fetal oximetry model as disclosed herein. In some cases, the developed simulated fetal oximetry model and/or an in vivo fetal oximetry model may compensate for one or more physio-optical influences that occur when performing transabdominal fetal oximetry. System 10 includes a cloud computing platform 11, a communication network 12, a computer 13, a display device 14, and a database 15. In many instances, communication network 12 is the Internet. The components of system 10 may be coupled together via wired and/or wireless communication links. In some instances, wireless communication of one or more components of system 10 may be enabled using short-range wireless communication protocols designed to communicate over relatively short distances (e.g., BLUETOOTH®, near field communication (NFC), radio-frequency identification (RFID), and Wi-Fi) with, for example, a computer or personal electronic device (e.g., tablet computer or smart phone) as described below.

Cloud computing platform 11 may be any cloud computing platform 11 configured to run a machine learning program and/or support a machine learning architecture such as TensorFlow. Exemplary cloud computing platforms include, but are not limited to, Amazon Web Service (AWS), Rackspace, and Microsoft Azure. Exemplary machine learning architectures include neural networks, artificial neural networks, Bayesian networks, and/or software or hardware that utilizes artificial intelligence.

Computer 13 may be configured to act as a communication terminal to cloud computing platform 11 via, for example, communication network 12 and may facilitate provision of the results machine learning calculations (e.g., training and/or testing of a simulated fetal oximetry model, tuning of a simulated fetal oximetry model, training and/or testing of a in vivo fetal oximetry model, and/or tuning of the in vivo fetal oximetry model) performed on cloud computing platform 11 to display device 155. Exemplary computers 13 include desktop and laptop computers, servers, tablet computers, personal electronic devices, mobile devices (e.g., smart phones), and the like. Exemplary display devices 155 are computer monitors, tablet computer devices, and displays provided by one or more of the components of system 10. In some instances, display device 155 may be resident in computer 13. Computer 13 may be communicatively coupled to database 15, which may be configured to store information (e.g., simulated optical inputs, simulated light transmission data sets, levels of a simulated fetal oximetry model, simulated and/or calculated fetal oximetry values, in vivo light transmission data sets, levels of an in vivo fetal oximetry model, model testing results, etc.), or inputs, used for machine learning and/or sets of instructions for computer 13 and/or cloud computing platform 11.

FIG. 1B is a block diagram of an exemplary system 100 for measuring in vivo light transmission data, measuring in vivo fetal oximetry values, and/or determining in vivo fetal oximetry values. In some embodiments, system 100 and/or a component thereof, such as computer 13, may be communicatively coupled to system 10, or a component thereof such as communication network 12 and/or cloud computing platform 11. The components of system 100 may be coupled together via wired and/or wireless communication links. In some instances, wireless communication of one or more components of system 100 may be enabled by using short-range wireless communication protocols designed to communicate over relatively short distances (e.g., BLUETOOTH®, near field communication (NFC), radio-frequency identification (RFID), and Wi-Fi) with, for example, a computer or personal electronic device (e.g., tablet computer or smart phone) as described below.

System 100 includes a light source 105 and a detector 160 that, at times, may be housed in a single housing, which may be referred to as a fetal probe 115. Light source 105 may include a single, or multiple light sources and detector 160 may include a single, or multiple detectors.

Light sources 105 may transmit light at light of one or more wavelengths, including NIR, into the pregnant mammal's abdomen. Typically, the light emitted by light sources 105 will be focused or emitted as a narrow beam to reduce spreading of the light upon entry into the pregnant mammal's abdomen. Light sources 105 may be, for example, a LED, and/or a LASER, a tunable light bulb and/or a tunable LED that may be coupled to a fiber optic cable. On some occasions, the light sources may be one or more fiber optic cables optically coupled to a laser and arranged in an array. In some instances, the light sources 105 may be tunable or otherwise user configurable while, in other instances, one or more of the light sources may be configured to emit light within a pre-defined range of wavelengths. Additionally, or alternatively, one or more filters (not shown) and/or polarizers may filter/polarize the light emitted by light sources 105 to be of one or more preferred wavelengths. These filters/polarizers may also be tunable or user configurable.

An exemplary light source 105 may have a relatively small form factor and may operate with high efficiency, which may serve to, for example, conserve space and/or limit heat emitted by the light source 105. In one embodiment, light source 105 is configured to emit light in the range of 770-850 nm. Exemplary flux ratios for light sources include but are not limited to a luminous flux/radiant flux of 175-260 mW, a total radiant flux of 300-550 mW and a power rating of 0.6 W-3.5 W.

Detector 160 may be configured to detect a light signal emitted from the pregnant mammal and/or the fetus via, for example, transmission and/or back scattering. Detector 160 may convert this light signal into an electronic signal, which may be communicated to a computer or processor and/or an on-board transceiver that may be capable of communicating the signal to the computer/processor. This emitted light might then be processed in order to determine how much light, at various wavelengths, passes through the fetus and/or is reflected and/or absorbed by the fetal oxyhemoglobin and/or de-oxyhemoglobin so that a fetal hemoglobin oxygen saturation level may be determined. This processing will be discussed in greater detail below. In some embodiments, detector 160 may be configured to detect/count single photons. At times, the optical signals detected by detector 160 and converted into an electronic signal corresponding to the detected optical signal may be referred to herein as measured, or in vivo, light transmission data and/or a detected signal.

Exemplary detectors include, but are not limited to, cameras, traditional photomultiplier tubes (PMTs), silicon PMTs, avalanche photodiodes, and silicon photodiodes. In some embodiments, the detectors will have a relatively low cost (e.g., $50 or below), a low voltage requirement (e.g., less than 100 volts), and non-glass (e.g., plastic) form factor. In other embodiments, (e.g., contactless pulse oximetry) a sensitive camera may be deployed to receive light emitted by the pregnant mammal's abdomen. For example, detector 160 may be a sensitive camera adapted to capture small changes in fetal skin tone caused by changes in cardiovascular pressure associated with fetal myocardial contractions. In these embodiments, detector 160 and/or fetal probe 115 may be in contact with the pregnant mammal's abdomen, or not, as this embodiment may be used to perform so-called contactless pulse oximetry. In these embodiments, light sources 105 may be adapted to provide light (e.g., in the visible spectrum, near-infrared, etc.) directed toward the pregnant mammal's abdomen so that the detector 160 is able to receive/detect light emitted by the pregnant mammal's abdomen and fetus. The emitted light captured by detector 160 may be communicated to computer 13 for processing to convert the images to a measurement of fetal hemoglobin oxygen saturation according to, for example, one or more of the processes described herein.

A fetal probe 115, light source 105, and/or detector 160 may be of any appropriate size and, in some circumstances, may be sized so as to accommodate the size of the pregnant mammal using any appropriate sizing system (e.g., waist size and/or small, medium, large, etc.). Exemplary lengths for a fetal probe 115 include a length of 4 cm-40 cm and a width of 2 cm-10 cm. In some circumstances, the size and/or configuration of a fetal probe 115, or components thereof, may be responsive to skin pigmentation of the pregnant mammal and/or fetus. In some instances, the fetal probe 115 may be applied to the pregnant mammal's skin via tape or a strap that cooperates with a mechanism (e.g., snap, loop, etc.) (not shown). In some instances, fetal probe 115 may act to pre-process or filter detected signals.

System 100 includes a number of optional independent sensors/probes designed to monitor various aspects of maternal and/or fetal health and may be in contact with a pregnant mammal. These probes/sensors are a NIRS adult hemoglobin probe 125, a pulse oximetry probe 130, a Doppler and/or ultrasound probe 135, and a uterine contraction measurement device 140. Not all embodiments of system 100 will include all of these components. In some embodiments, system 100 may also include an electrocardiography (ECG) machine (not shown) that may be used to determine the pregnant mammal's and/or fetus's heart rate and/or an intrauterine pulse oximetry probe (not shown) that may be used to determine the fetus's heart rate. The Doppler and/or ultrasound probe 135 may be configured to be placed on the abdomen of the pregnant mammal and may be of a size and shape that approximates a silver U.S. dollar coin and may provide information regarding fetal position, orientation, and/or heart rate. Pulse oximetry probe 130 may be a conventional pulse oximetry probe placed on pregnant mammal's hand and/or finger to measure the pregnant mammal's hemoglobin oxygen saturation. NIRS adult hemoglobin probe 125 may be placed on, for example, the pregnant mammal's 2nd finger and may be configured to, for example, use near infrared spectroscopy to calculate the ratio of adult oxyhemoglobin to adult de-oxyhemoglobin. NIRS adult hemoglobin probe 125 may also be used to determine the pregnant mammal's heart rate.

Optionally, system 100 may include a uterine contraction measurement device 140 configured to measure the strength and/or timing of the pregnant mammal's uterine contractions. In some embodiments, uterine contractions will be measured by uterine contraction measurement device 140 as a function of pressure (e.g., measured in e.g., mmHg) over time. In some instances, the uterine contraction measurement device 140 is and/or includes a tocotransducer, which is an instrument that includes a pressure-sensing area that detects changes in the abdominal contour to measure uterine activity and, in this way, monitors frequency and duration of contractions.

In another embodiment, uterine contraction measurement device 140 may be configured to pass an electrical current through the pregnant mammal and measure changes in the electrical current as the uterus contracts. Additionally, or alternatively, uterine contractions may also be measured via near infrared spectroscopy using, for example, light received/detected by detector 160 because uterine contractions, which are muscle contractions, are oscillations of the uterine muscle between a contracted state and a relaxed state. Oxygen consumption of the uterine muscle during both of these stages is different and these differences may be detectable using NIRS.

Measurements and/or signals from NIRS adult hemoglobin probe 125, pulse oximetry probe 130, Doppler and/or ultrasound probe 135, and/or uterine contraction measurement device 140 may be communicated to receiver 145 for communication to computer 13 and display on display device 155 and, in some instances, may be considered secondary signals. As will be discussed below, measurements provided by NIRS adult hemoglobin probe 125, pulse oximetry probe 130, a Doppler and/or ultrasound probe 135, uterine contraction measurement device 140 may be used in conjunction with fetal probe 115 to isolate a fetal pulse signal and/or fetal heart rate from a maternal pulse signal and/or maternal heart rate. Receiver 145 may be configured to receive signals and/or data from one or more components of system 100 including, but not limited to, fetal probe 115, NIRS adult hemoglobin probe 125, pulse oximetry probe 130, Doppler and/or ultrasound probe 135, and/or uterine contraction measurement device 140. Communication of receiver 145 with other components of system may be made using wired or wireless communication.

In some instances, one or more of NIRS adult hemoglobin probe 125, pulse oximetry probe 130, a Doppler and/or ultrasound probe 135, uterine contraction measurement device 140 may include a dedicated display that provides the measurements to, for example, a user or medical treatment provider. It is important to note that not all of these probes may be used in every instance. For example, when the pregnant mammal is using fetal probe 115 in a setting outside of a hospital or treatment facility (e.g., at home or work) then, some of the probes (e.g., NIRS adult hemoglobin probe 125, pulse oximetry probe 130, a Doppler and/or ultrasound probe 135, uterine contraction measurement device 140) of system 100 may not be used.

In some instances, receiver 145 may be configured to process or pre-process received signals so as to, for example, make the signals compatible with computer 13 (e.g., convert an optical signal to an electrical signal), improve signal to noise ratio (SNR), amplify a received signal, etc. In some instances, receiver 145 may be resident within and/or a component of computer 13. In some embodiments, computer 13 may amplify or otherwise condition the received detected signal so as to, for example, improve the signal-to-noise ratio.

Receiver 145 may communicate received, pre-processed, and/or processed signals to computer 13. Computer 13 may act to process the received signals, as discussed in greater detail below, and facilitate provision of the results to a display device 155. Exemplary computers 13 include desktop and laptop computers, servers, tablet computers, personal electronic devices, mobile devices (e.g., smart phones), and the like. Exemplary display devices 155 are computer monitors, tablet computer devices, and displays provided by one or more of the components of system 100. In some instances, display device 155 may be resident in receiver 145 and/or computer 13. Computer 13 may be communicatively coupled to database 170, which may be configured to store information regarding physiological characteristic and/or combinations of physiological characteristic of pregnant mammals and/or their fetuses, impacts of physiological characteristic on light behavior, information regarding the calculation of hemoglobin oxygen saturation levels, calibration factors, calibration formulas, calibration curves, and so on.

In some embodiments, a pregnant mammal may be electrically insulated from one or more components of system 100 by, for example, an electricity isolator 120. Exemplary electricity insulators 120 include circuit breakers, ground fault switches, and fuses.

In some embodiments, system 100 may include an electro-cardiogram (ECG) machine 175 configured to ascertain characteristics of the pregnant mammal's heart rate and/or pulse and/or measure same. These characteristics may be used as, for example, a secondary signal and/or maternal heart rate signal as disclosed herein.

In some embodiments, system 100 may include a ventilatory/respiratory signal source 180 that may be configured to monitor the pregnant mammal's respiratory rate and provide a respiratory signal indicating the pregnant mammal's respiratory rate to, for example, computer 13. Additionally, or alternatively, ventilatory/respiratory signal source 180 may be a source of a ventilatory signal obtained via, for example, cooperation with a ventilation machine. Exemplary ventilatory/respiratory signal sources 180 include, but are not limited to, a carbon dioxide measurement device, a stethoscope and/or electronic acoustic stethoscope, a device that measures chest excursion for the pregnant mammal, and a pulse oximeter. A signal from a pulse oximeter may be analyzed to determine variations in the PPG signal that may correspond to respiration for the pregnant mammal. Additionally, or alternatively, ventilatory/respiratory signal source 180 may provide a respiratory signal that corresponds to a frequency with which gas (e.g., air, anesthetic, etc.) is provided to the pregnant mammal during, for example, a surgical procedure. This respiratory signal may be used to, for example, determine a frequency of respiration for the pregnant mammal.

In some embodiments, system 100 may include a timestamping device 185. Timestamping device 185 may be configured to timestamp a signal provided by, for example, fetal probe 115, Doppler/ultrasound probe 135, pulse oximetry probe 130, NIRS adult hemoglobin probe, uterine contraction measurement device 140, ECG 175, and/or ventilatory/respiratory signal source 180 with a timestamp that represents, for example, an event (e.g., time, or t, =0, 10, 20, etc.) and/or chronological time (e.g., date and time). Timestamping device 185 may time stamp a signal via, for example, introducing a ground signal into system 100 that may simultaneously, or nearly simultaneously, interrupt or otherwise introduce a stamp or other indicator into a signal generated by one or more of, for example, fetal probe 115, Doppler/ultrasound probe 135, pulse oximetry probe 130, NIRS adult hemoglobin probe, uterine contraction measurement device 140, ECG 175, and/or ventilatory/respiratory signal source 180. Additionally, or alternatively, timestamping device 185 may time stamp a signal via, for example, introducing an optical signal into system 100 that may simultaneously, or nearly simultaneously, interrupt or otherwise introduce a stamp or other indicator into a signal generated by one or more of, for example, fetal probe 115, pulse oximetry probe 130, NIRS adult hemoglobin probe, uterine contraction measurement device 140. Additionally, or alternatively, timestamping device 185 may time stamp a signal via, for example, introducing an acoustic signal into system 100 that may simultaneously, or nearly simultaneously, interrupt or otherwise introduce a stamp or other indicator into a signal generated by one or more of, for example, fetal probe 115, Doppler/ultrasound probe 135, and/or ventilatory/respiratory signal source 180.

A timestamp generated by timestamping device 185 may serve as a simultaneous, or nearly simultaneous starting point, or benchmark, for the processing, measuring, synchronizing, correlating, and/or analyzing of a signal from, for example, fetal probe 115, Doppler/ultrasound probe 135, pulse oximetry probe 130, NIRS adult hemoglobin probe, uterine contraction measurement device 140, ECG 175, and/or ventilatory/respiratory signal source 180. In some instances, a time stamp may be used to relate and/or synchronize two or more signals generated by, for example, fetal probe 115, Doppler/ultrasound probe 135, pulse oximetry probe 130, NIRS adult hemoglobin probe, uterine contraction measurement device 140, ECG 175, and/or ventilatory/respiratory signal source 180 so that, for example, they align in the time domain.

FIG. 1C is a block diagram of an exemplary fetal oximetry probe system 117 that, on some occasions, may be used with system 100 in addition to, or instead of, fetal oximetry probe 115. Fetal oximetry probe system 117 includes a first light source/detector system 107 and a second light source/detector system 167 housed within a housing 127 that may be configured to enable use of the fetal oximetry probe system 117. First light source/detector system 107 and second light source/detector system 167 may be configured in a manner that is similar to, or different from, one another. For example, first light source/detector system 107 may be configured as a frequency-domain measurement system and second light source/detector system 167 may be configured as a near infrared spectroscopy system. Additionally, or alternatively, first light source/detector system 107 may be configured as a system that measures a time of flight of photons projected into the pregnant mammal's abdomen and returning to one or more detectors like detectors 160. On some occasions, the frequency-domain and/or time of flight measurements may be used to, for example, determine optical properties (e.g., scattering and/or absorption coefficients) of maternal and/or fetal tissue. Relative positions of the first light source/detector system 107 and second light source/detector system 167 may be known so that, for example, data received via by first light source/detector system 107 may be used to validate and/or further analyze data received via second light source/detector system 167.

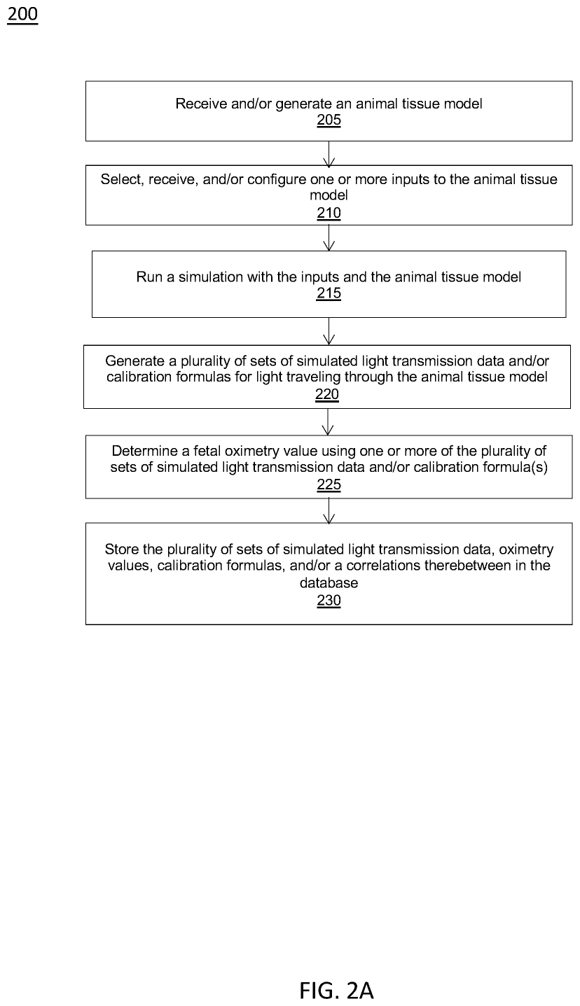

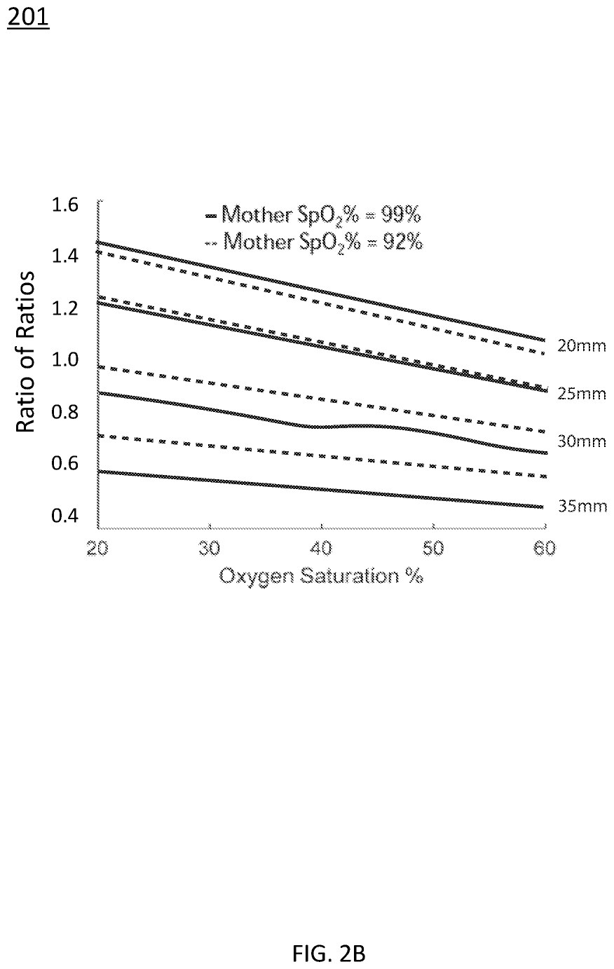

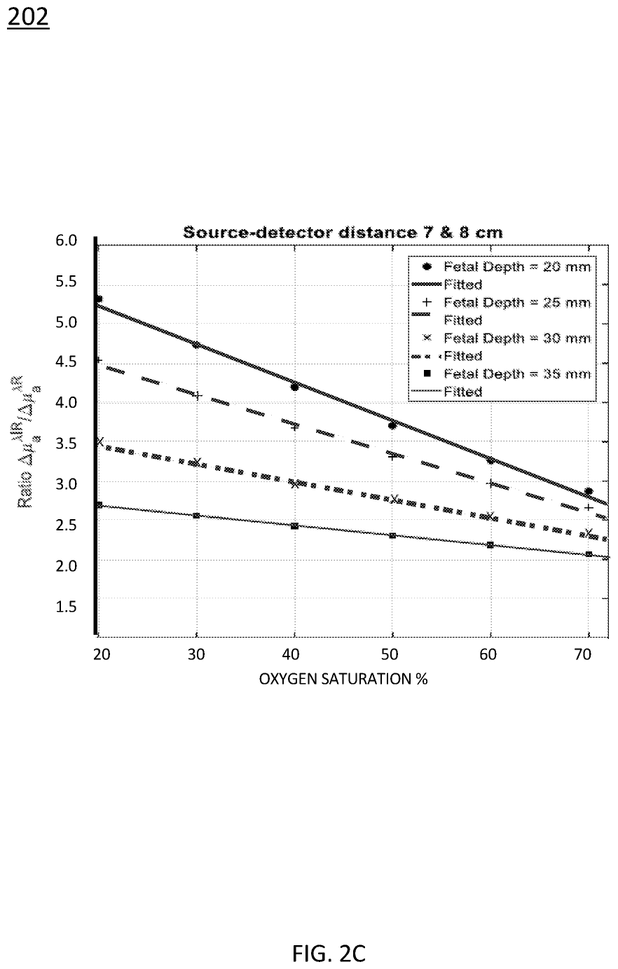

FIG. 2A is a flowchart showing an exemplary process 200 for generating a plurality of sets of simulated light transmission data and corresponding oximetry values using a computer-generated, or simulated, model of animal tissue. Process 200 may be executed by, for example, system 100, 10, and/or components thereof.

Initially, in step 205, a two and/or three-dimensional model of a portion of animal tissue may be generated and/or received. When a model is generated, it may be generated using one or more parameters of, for example, a pregnant mammal and/or a fetus. Often times, the model generated and/or received in step 205 includes a plurality of layers (at least one maternal and one fetal) and the layers of the model may each have, and/or be associated with, one or more optical properties such as absorption and/or reflection characteristics, blood saturation characteristics, and/or width. In some cases, the one or more optical properties of the modeled tissue may be dictated by chemical properties of the tissue such as lipid content, water content, density, and/or tissue type. Additionally, or alternatively, one or more properties of the modeled tissue may correspond to geometrical parameters for the modeled tissue such as width, depth, orientation, and/or how different types of tissue may interact with one another to transmit, reflect, scatter, and/or absorb light. Additionally, or alternatively, other parameters such as tissue composition (e.g., lipid content, water content, muscle cell content, etc.), noise, ambient light, scattering coefficients of the modeled tissue and/or layers thereof, absorption coefficients of the modeled tissue and/or layers thereof, an overall thickness of the model, fetal depth, maternal skin color, maternal skin melanin content, fetal skin color, fetal skin melanin content, maternal and/or fetal tissue layer composition (e.g., skin, adipose, and/or muscle tissue) and/or relative thicknesses of the tissue layers for the maternal and/or fetal tissue.

In some cases, execution of step 205 may also include receipt and/or selection of parameters or rules for the model, some of which may be machine learning inputs and/or optical properties used to generate and/or modify one or more layers of the model. When process 200 is executed multiple (e.g., hundreds, thousands, and/or hundreds of thousands) times, one or more aspects and/or parameters of the model received and/or generated in step 205 may be altered and/or changed so that, for example, a database of results of executing process 200 may be generated that show results of execution of step 205 for different model parameters.

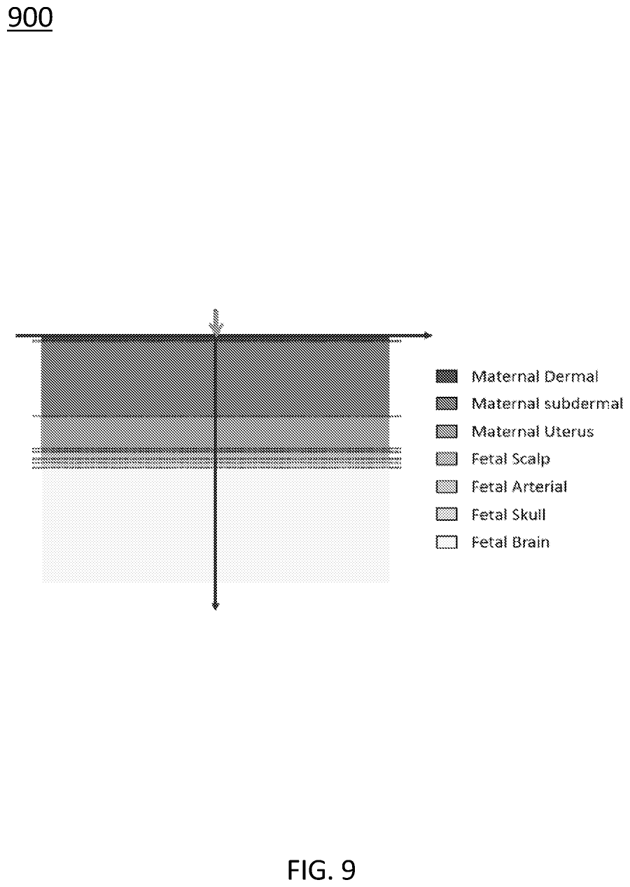

FIG. 9 provides an image 900 of an exemplary seven-layer two-dimensional model 900 of a pregnant mammal's abdomen and her fetus that may be received and/or generated via execution of step 205. The seven layers of two-dimensional model 900 are 1) maternal dermal, 2) maternal subdermal, 3) maternal uterus, 4) fetal scalp, 5) fetal arterial, 6) fetal skull, and 7) fetal brain. Each of these layers may have different optical properties based on, for example, characteristics (e.g., wavelength, intensity, modulations, etc.) of light incident thereon, tissue layer composition, tissue layer thickness, and/or tissue layer geometry.

In step 210, one or more inputs to and/or parameters for the animal tissue model of step 205 may be designed, calculated, selected, received, and/or configured. In some embodiments, individual inputs and/or parameters may be randomly, pseudo randomly, and/or systematically designed, calculated, selected, received, and/or configured according to, for example, one or more methodologies and/or algorithms. The individual inputs and/or parameters may be systematically designed, calculated, selected, received, and/or configured according to, for example, a physiologically appropriate distribution (e.g., likelihood of occurrence within a population) assigned that may be associated with the individual input and/or parameter. Additionally, or alternatively, individual inputs and/or parameters may be systematically designed, calculated, selected, received, and/or configured to accommodate an input and/or particular parameter of interest or relevance to a user.

Exemplary inputs and/or parameters for step 210 include optical inputs/parameters such as simulated light wavelength(s), simulated light intensity, modulation parameters (e.g., a duration of successive light pulses) for incident simulated light, and/or multiplexing parameters (e.g., a duration and/or wavelength of successive light pulses) for incident simulated light. In some cases, the simulated optical inputs may dictate parameters for the simulation of behavior of infra-red and/or near infra-red light as it travels through a model of step 205.

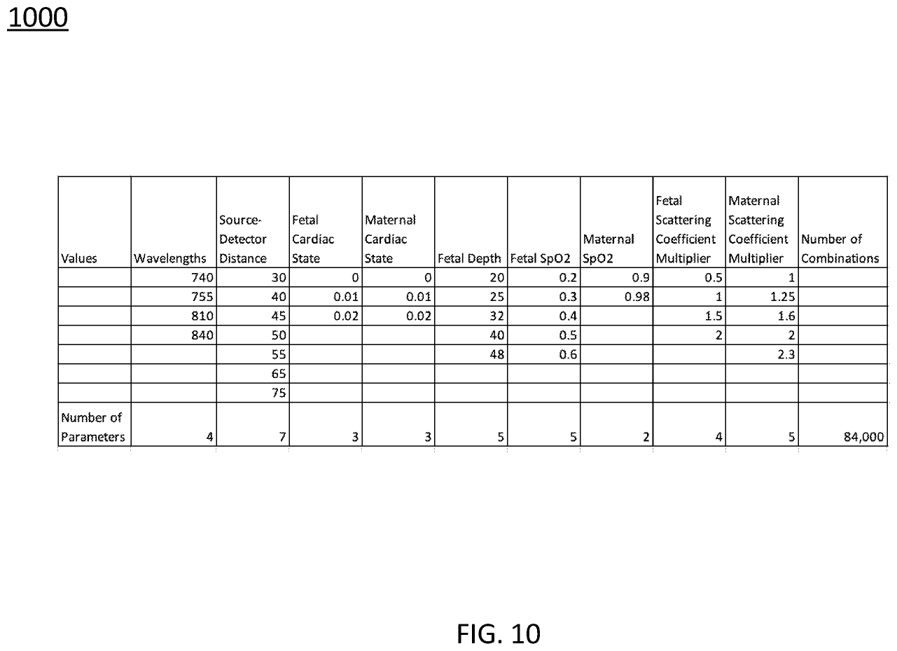

FIG. 10 provides a table 1000 of exemplary inputs and/or parameters that may be designed, calculated, selected, received, and/or configured in step 210, such as exemplary values for a wavelength of simulated light to be projected into the model of step 205, a distance between the source of the simulated light and a detector that may “detect” the simulated light, fetal cardiac state, maternal cardiac state, fetal depth, fetal SpO2, maternal SpO2, fetal scattering coefficient multiplier, and maternal scattering coefficient multiplier.

Additionally, or alternatively, exemplary inputs and/or parameters for step 210 may include fetal and maternal cross correlation with heartbeats, fetal heart rate, maternal heart rate, fetal and/or maternal DC level, maternal SpO2, maternal bold oxygenation values, maternal tissue oxygenation values, maternal SpO2 values, maternal venous hemoglobin oxygen saturation values, fetal venous hemoglobin oxygen saturation values, fetal depth normalization ratios, correlation amplitudes, time of flight for photons traveling through the model, fast Fourier transforms (FFTs) and/or R values. At times, exemplary inputs and/or parameters for step 210 may include of one or more time series waveforms with variable fetal (100 to 240 BPM) and/or maternal (50 to 12 BPM) heartrates, amplitudes, and/or phases between them.

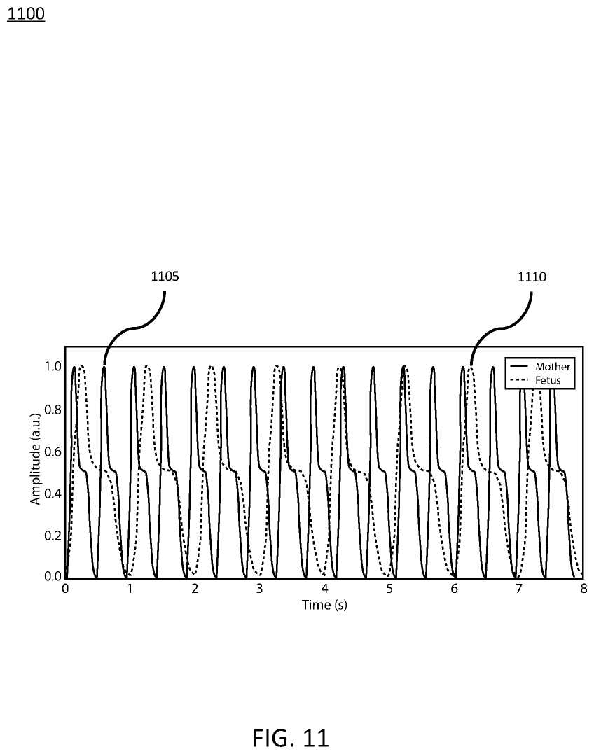

Additionally, or alternatively, exemplary inputs and/or parameters for step 210 may include one or more photoplethysmogram (PPG) signal(s) and/or a modulated PPG signal(s) that may simulate cardiac cycles for the pregnant mammal and/or fetus. An exemplary PPG modulated signal may have a variable 1% to 2% change in systolic blood volume for the pregnant mammal and/or fetus over time. FIG. 11 provides an exemplary graph 1100 that plots simulated fetal and maternal PPG signals over time in seconds, wherein a PPG signal for the mother/pregnant mammal 1105 is shown in black and a PPG signal for the fetus 1110 is shown in grey. In some embodiments, noise and/or a confounding factor may be added to the PPG signal for the fetus 1110 and/or pregnant mammal 1105 as part of, for example, execution of a perturbation analysis using the model of step 205. A result of the perturbation analysis may be incorporated into, for example, generation of additional models and/or machine learning and/or model training as, for example, described herein.

Additionally, or alternatively, exemplary inputs and/or parameters for step 210 may include oximetry values for the pregnant mammal and/or fetus, such as a percent of hemoglobin saturated with oxygen (e.g., hemoglobin oxygen saturation percent or level), a relative oximetry value, and/or a ratio of oxygenated hemoglobin compared with deoxygenated hemoglobin.

Additionally, or alternatively, exemplary inputs and/or parameters for step 210 may include various parameters (e.g., sensitivity, area over which simulated photons and/or light signals are detected, etc.) for simulated photodetector(s) that may be used to “detect” light as it travels through and/or emanates from the model of step 205 in order to, for example, simulate an operation of different types of photodetectors and/or different conditions (e.g., age, hours of use, type, level of sensitivity size, power drawn detector sensitivity, lag times, light source characteristics, and/or errors or noise that may be introduced into a signal when particular equipment is used) under which the photodetector may be operating. Additionally, or alternatively, exemplary inputs and/or parameters for step 210 may include various parameters (e.g., intensity, wavelength, duration of light pulses, etc.) for simulated light source(s) that may be used to “emit” light into the model of step 205 in order to, for example, simulate an operation of different types of light sources and/or different conditions (e.g., age, hours of use, type, level of sensitivity size, and/or power drawn) under which the light source may be operating. Additionally, or alternatively, exemplary inputs and/or parameters for step 210 may include various classifiers and/or loss functions for the model and/or inputs. Additionally, or alternatively, exemplary inputs and/or parameters for step 210 may include features for use with different machine learning architectures and/or computing equipment that may have, for example, varying computational capabilities and/or processing rates.

Next, in step 215, one or more simulation(s) using the model and simulated optical inputs of steps 205 and 210, respectively, may be run, or executed, wherein simulated light is transmitted through the model and “detected” by a simulated photodetector, thereby generating a set of simulated light transmission data and/or calibration formulas for simulated light traveling through the animal tissue model (step 220). In some cases, a set of simulated light transmission data may correspond to simulated light being transmitted through the model for a period of time (e.g., 15, 30, or 60 seconds; 1, 5, or 10 minutes). Steps 215 and/or 220 may be executed by, for example, a computer or processor such as cloud computing platform 11 and/or computer 13 with, for example, modeling and/or simulation software such as Monte Carlo simulations and/or NIRFAST calculations. In some embodiments, steps 215 and 220 may be executed a plurality (e.g., 50,000; 100,000; 500,000; 1,000,000; 5,000,000) of times thereby generating a plurality of sets of simulated light transmission data.

At times, the calibration formulas may relate to, for example, a ratio of ratios (R) and/or an optical density of tissue with fetal oximetry values. R may be calculated for the fetus according to, for example, Equation 3, below:

$$R = \frac{A C / D C ( λ 1 )}{A C / D C ( λ 2 )} \qquad Equation 3$$

Where AC corresponds to a photo-plethysmography (PPG) pulse amplitude at end diastole and DC corresponds to corresponds to a PPG pulse amplitude during systole. Additionally, or alternatively, R may be calculated via equation 4, below:

$$R = \frac{{[ ( {I}_{D} - {I}_{S} ) / {I}_{S} ]}_{1}}{{[ ( {I}_{D} - {I}_{S} ) / {I}_{S} ]}_{2}} \qquad Equation 4$$

Where I_{D} is a PPG pulse amplitude at end diastole and I_{S} is a PPG pulse amplitude during systole and the numerator of Equation 4 corresponds to I_{D} and I_{S} values for a first wavelength of light and the denominator Equation 4 corresponds to I_{D} and I_{S} values for a second wavelength of light.

On some occasions, a plurality (e.g., 100-100,000) of calibration formulas may be generated that incorporate various factors and/or inputs regarding light (e.g., wavelength and/or intensity) that may be simulated to travel through the animal model; geometrical properties (e.g., distance light travels (e.g., fetal depth and/or modeled layer thickness), a shape of tissue within the animal tissue model, and/or a thickness of tissue within the animal tissue model; optical properties of the animal model (e.g., scattering coefficient and absorption coefficient); time of flight for photons traveling through the animal model, and/or physiological properties of the modeled maternal and/or fetal tissue (e.g., maternal hemoglobin oxygen saturation levels and/or skin color). In some embodiments, step 215 and/or 220 may be executed by, for example, performing Monte Carlo simulations and NIRFAST calculations using the model of step 205 and/or the simulated optical signal inputs and/or the oximetry value inputs of step 210 to model and/or predict behavior (e.g., transmission, absorption, and/or scattering) of an optical signal generated using the optical signal inputs as it travels through the animal tissue model.

On some occasions, execution of step 220 may include determining one or more calibration formulas for simulated light traveling through the model. A calibration formula may correspond to how simulated light travels through a model and may be used to, for example, calibrate simulated light as it travels through a model so that one or more sets of simulated light transmission data may be used to calculate a simulated fetal oximetry value (step 225) using, for example, the Beer Lambert Law or a modified version of the Beer Lambert Law as explained above using Equations 1 and 2.

FIG. 2B is a graph 201 that plots exemplary relationships, or calibration formulas, between a ratio of ratios (R) and a hemoglobin oxygen saturation percentage of a fetus for various fetal depths along with best fit curves that may be calculated/determined via process 200. More particularly, graph 201 plots how a ratio of ratios (R) and, in particular an R value for a fetus, may be correlated with a simulated hemoglobin oxygen saturation percentage of a fetus for modeled fetal depths of 20 mm, 25 mm, 30 mm, and 35 mm when the modeled maternal SpO2% is 99% (solid line) or 92% (broken line) along with a corresponding best-fit curve for each modeled fetal depth. A formula defining the best-fit line may, in some cases, be a calibration formula for use with, for example, one or more of the models and/or fetal oximetry calculations disclosed herein. On some occasions, the best-fit line(s) of graph 201 may be calibration curve(s).

FIG. 2C is a graph 202 that plots exemplary relationships between a ratio of a change in an absorption coefficient for a modeled and/or simulated a first (e.g., infrared) wavelength of light divided by a ratio of a change in a modeled and/or simulated absorption coefficient for a second (e.g., red) wavelength of light and a calculated hemoglobin oxygen saturation percentage of a fetus for modeled fetal depths of 20 mm, 25 mm, 30 mm, and 35 mm along with a corresponding best-fit curve for each modeled fetal depth that may be calculated/determined via process 200. A formula defining the best-fit line(s) of graph 202 may, in some cases, be a calibration formula for use with, for example, one or more of the models disclosed herein. On some occasions, the best-fit line(s) of graph 202 may be calibration curve(s) that are an exemplary output of execution of process 200 and/or step 220.

Following step 225, the plurality of simulated light transmission data sets, oximetry values, calibration formulas, and/or correlations between the set(s) of simulated light transmission data and/or calibration formula and it's respective oximetry value may be stored in a database (step 230) like database 15 and/or 170. On some occasions, the data stored in step 230 may be used as simulation parameters and/or inputs for one or more machine learning processes and/or the development of one or more algorithms disclosed herein.

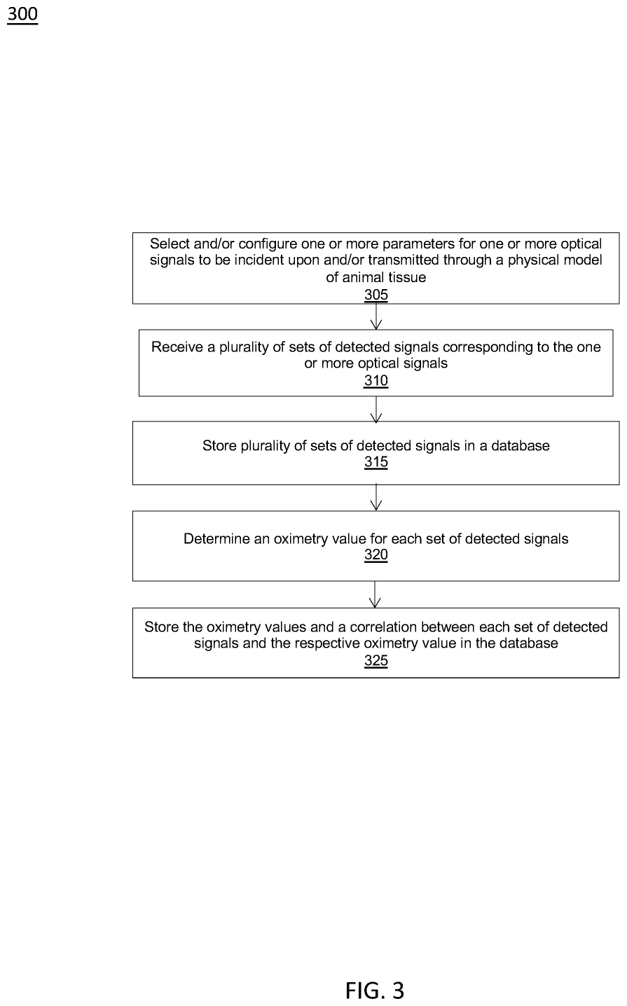

FIG. 3 is a flowchart showing an exemplary process 300 for generating a plurality of sets of simulated light transmission data and corresponding oximetry values using light transmitted through a physical model of animal tissue. Portions of process 300 may be executed by, for example, system 100, 10, and/or components thereof.

In step 305, one or more inputs and/or parameters for the generation of one or more optical signals to be incident upon and/or transmitted a physical model of animal tissue may be selected, received, and/or configured. Exemplary optical inputs include, but are not limited to, light wavelength, intensity, modulation of the light (e.g., a duration of successive light pulses), and/or a range of wavelengths. In many cases, the optical inputs will be for the generation of infra-red and/or near infra-red light. The physical model of tissue may comprise one or more layers that have the same or different optical properties. The physical model may be made from one or layers of for example, gels, aqueous solutions, and lipids.

The optical signals selected, generated, and/or configured in step 305 may then be projected into the physical model of animal tissue by one or more light sources (e.g., light source 105) and detected by one or more photodetectors (e.g., detector 160), which may communicate a signal (e.g., analog or digital) corresponding to the detected light/optical signal to, for example, a processor or circuit may then be received from the photodetector (step 310). In some cases, the detected signals may correspond to light being transmitted through the physical model for a period of time (e.g., 15, 30, or 60 seconds; 1, 5, or 10 minutes). A result of execution of step 310 may be the generation of a set of simulated light transmission data. Step 310 may be executed a plurality (e.g., 50,000; 100,000; 500,000; 1,000,000; 5,000,000) of times thereby generating a plurality of sets of simulated light transmission data. The plurality of sets of detected signals may then be stored in a database (step 315) like database 15 and/or 170. In some instances, the sets of detected signals may be correlated with the physical model and/or a characteristic of the physical model in step 315.

In step 320, an oximetry value for each set of detected signals may be determined and/or received. The oximetry value may be, for example, a maternal hemoglobin oxygen saturation level, a maternal tissue oxygenation level, a fetal hemoglobin oxygen saturation level, and/or a fetal tissue oxygenation level. When the oximetry values are hemoglobin oxygen saturation levels, the oximetry values may be determined via, for example, the Beer Lambert Law or a modified version of the Beer Lambert Law as explained above using Equations 1 and 2. When the oximetry values are tissue oxygen saturation levels, the oximetry values may be determined via, for example, diffuse optical tomography (DOT) or another tissue oxygen saturation determination technique. Following step 320, the oximetry values and/or correlations between each set of detected signals (which may also be referred to herein as simulated light transmission data) and it's respective oximetry value may be stored in a database (step 325) like database 15 and/or 170.

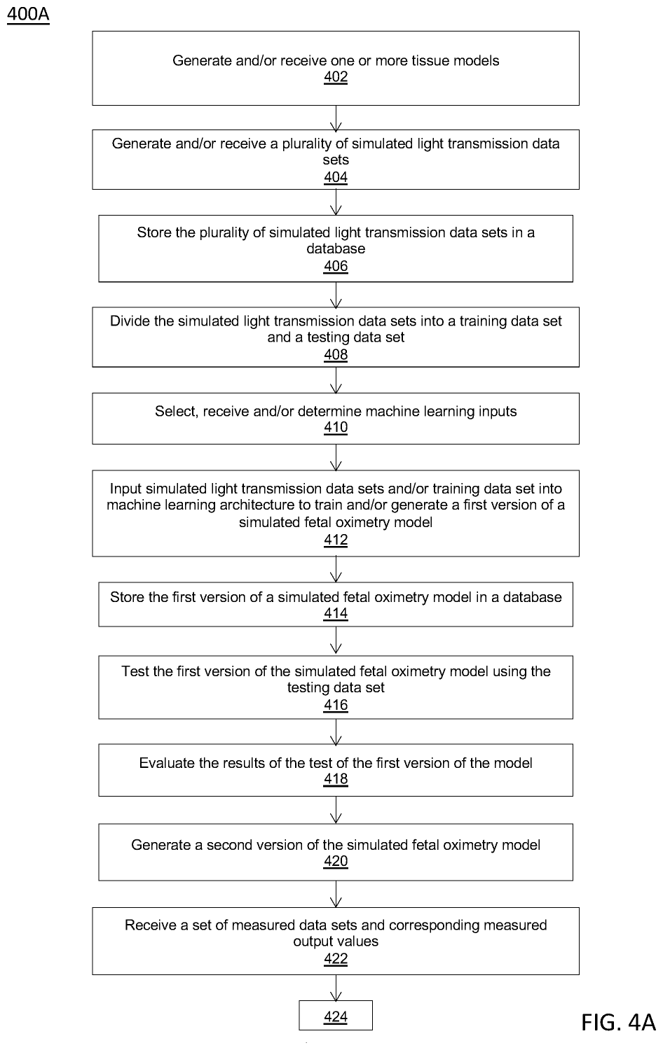

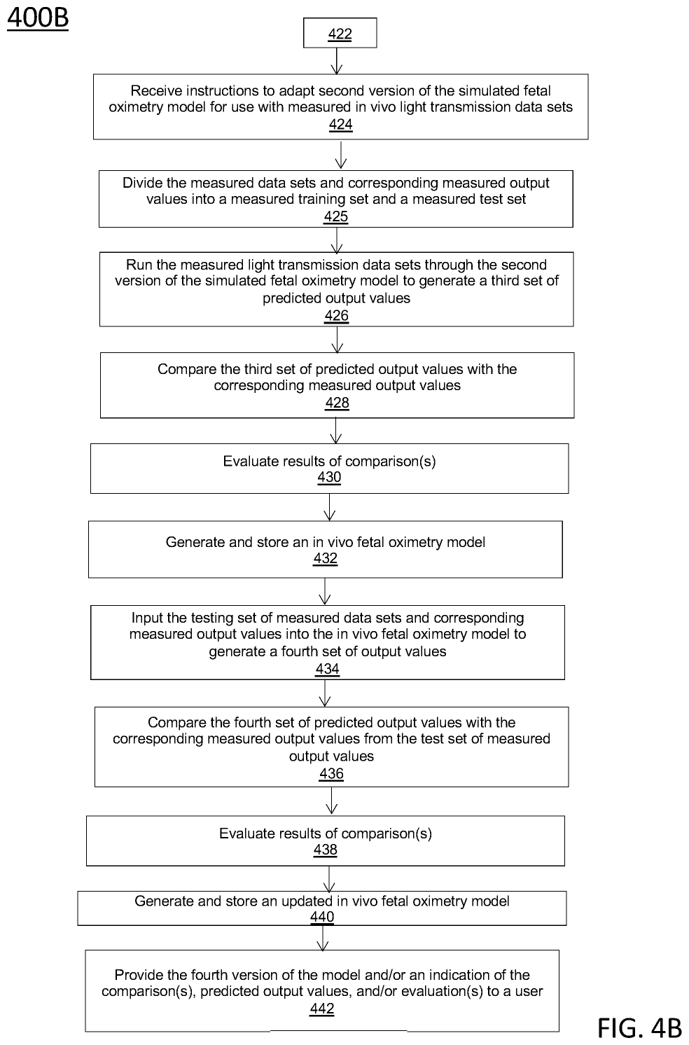

FIGS. 4A and 4B provide a flowchart (over two pages) showing an exemplary process 400 for developing an oximetry model that may be used to accurately calculate oximetry values for a target tissue within a body, such as a fetus in-utero. Process 400 may be executed by, for example, system 100, 10, and/or components thereof and, in some cases, execution of process 1400 may incorporate execution of one or more additional processes and/or process steps disclosed herein. In some embodiments, models (e.g., simulated fetal oximetry models) generated via execution of process 400 may include tree-based models or ensembles of layered and/or tree-based models. Additionally, or alternatively, models (e.g., simulated fetal oximetry models) generated via execution of process 400 may incorporate K-fold cross-validation to, for example, generate the expected error, receiver operating characteristic (ROC), and/or area under the curve (AUC) values for the model.

Initially, in step 402, a tissue model such as the tissue model(s) generated via execution of process 200 and/or 300. The tissue model may be a two and/or three-dimensional model of a portion of an animal (e.g., human) body with one or more layers of tissue.

In step 404, a plurality (e.g., 500,000; 1,000,000; 5,000,000) of simulated light transmission data sets may be received and/or generated via, or example, one or more processes disclosed herein. The light transmission data sets may be simulations of one or more optical signal(s), that may be emitted by a simulated light source, traveling, over a period of time (e.g., 10 s, 30 s, 60 s, 5 minutes, etc.), through one or more models received and/or generated in step 402 and being “detected” by a detector like detector 160.

In some embodiments, execution of step 404 may include running a plurality (e.g., 50-50,000) of experiments and/or simulations with different inputs (e.g., fetal and maternal cross correlation with heartbeats, DC level, maternal SpO2, normalization ratios, fetal depth, and/or maternal optical scattering properties) and/or different machine learning architectures. In some cases, different classifiers and/or loss functions may be used to generate a large number (e.g., 2-5 million) of data sets from which fetal oximetry values (e.g., fetal SpO2, fetal tissue oxygen saturation, etc.) may be calculated via, for example, execution of process 400 and/or a step thereof. Additionally, or alternatively, execution of step 404 may include running a plurality (e.g., 50-50,000) of experiments and/or simulations with different inputs that pertain to features of equipment (e.g., detector sensitivity, lag times, light source characteristics, errors or noise that may be introduced into a signal when particular equipment is used, etc.) that may be used and/or present when taking in vivo light transmission and/or fetal oximetry measurements are taken and/or observed.

The received and/or generated simulated light transmission data sets may then be stored in a database like database 15 and/or 170 (step 406). Optionally, in step 408, the simulated light transmission data sets may then be divided into a training set (e.g., 60%, 70%, or 80% of the data sets) and a testing set (e.g., 40%, 30%, or 20% of the data sets).

Optionally, in step 410, inputs to the machine learning architecture and/or software program for determining fetal oximetry values may be selected. Exemplary inputs include, but are not limited to, fetal depth, fetal heart rate, maternal heart rate, equipment characteristics, background noise characteristics, maternal geometrical characteristics, maternal physiological characteristics, fetal geometrical characteristics, fetal physiological characteristics and/or maternal oximetry values (e.g., SpO2 and/or DC oxygen saturation levels). In some embodiments, one or more inputs may be received from a component of system 100 such as ECG 175, Doppler/ultrasound probe 135, pulse oximetry probe 130, NIRS adult hemoglobin probe 125, and/or ventilator/ventilatory signal device. In some instances, input values and/or parameters may be normalized to, for example, standard mean and/or variance values, such as zero mean and unit variance, and, in some instances, may be combined into composite features that are then input into the machine learning architecture.

In some cases, the machine learning architecture disclosed herein may be a deep learning network architecture that may include convolutional nets and engineered feature layers. Additionally, or alternatively, the machine learning architecture may be a neural network, an artificial neural network, a Bayesian network, and/or software or hardware that utilizes artificial intelligence.

In some embodiments, execution of step 410 may include inputs that define and/or set parameters for down sampling and/or activating one or convolutional layers of the machine learning architecture and/or a model (e.g., a simulated fetal oximetry model) generated by the machine learning architecture. In some cases, execution of step 410 may also include adding one or more engineered features, bias, and/or classifier layers to the machine learning architecture and/or a model (e.g., a simulated fetal oximetry model) generated by the machine learning architecture.

In some embodiments, execution of step 410 may include selection of one or more types of outputs that may be incorporated into the machine learning architecture. Exemplary outputs include predicted fetal oximetry (e.g., SpO2 and/or fetal tissue oxygen saturation) values and a binary fetal hypoxia, fetal hypoxemia, fetal non-hypoxia, and/or fetal non-hypoxemia (e.g., fetal SpO2 above/below 30%) indication.

In step 412, the simulated light transmission data sets and/or training data set (when step 408 is executed) may be input into the machine learning architecture to generate and/or train a first version of a fetal oximetry model that may be configured to, for example, predict a first set of outputs (e.g., fetal SpO2 values, fetal tissue oxygen saturation, and/or fetal hypoxemia or non-hypoxemia determinations) using the simulated light transmission data sets and/or training data set. The first version of the simulated fetal oximetry model may include a plurality of layers and/or functions and, in some cases, may include one or more small layered network(s), sub-networks, and/or a Support Vector Machine. In some embodiments, execution of step 412 may include communication of the machine learning inputs and/or machine learning architecture characteristics (e.g., name, capacity, processing speeds, processor configuration, etc.) to, for example, a machine learning computer platform and/or neural network such as a machine learning platform resident on/within cloud computing platform 11. In step 414, the first version of the simulated fetal oximetry model may be stored in a database such as database 15 and/or 170.

Optionally, in step 416, the first version of the simulated fetal oximetry model and/or first set of outputs may be tested using, for example, the testing data set from step 408. The results of the testing may then be evaluated (step 418) and used to modify, iterate, and/or update the first version of the simulated fetal oximetry model thereby generating a second version of the simulated fetal oximetry model (step 420) via, for example, training and/or tuning the first version of the simulated fetal oximetry algorithm using the machine learning architecture and the testing data. The second version of the simulated fetal oximetry model may be used to predict a second set of outputs. In some embodiments, the second version of the simulated fetal oximetry model may be similar, or identical to, the first version of the fetal oximetry model. In other embodiments, the second version of the simulated fetal oximetry model may be more precise and/or accurate than the first version of the simulated fetal oximetry model.

Then, in step 422, a set of measured, or actual, in vivo light transmission data sets and corresponding output data (e.g., fetal oximetry values such as fetal SpO2 values, fetal venous hemoglobin oxygen saturation values, fetal DC values, and/or fetal tissue oxygenation saturation values) may be received. The in vivo light transmission data sets may be received from, for example, a fetal oximetry probe such as fetal oximetry probe 115 and/or fetal oximetry probe system 117 and each corresponding output data/oximetry value may be calculated using, for example, a corresponding in vivo light transmission data set received in step 422. In one embodiment, the set of measured in vivo light transmission data sets and corresponding measured output data may include 200-100,000 datasets/output values that, in some cases, may be correlated with additional information such as one or more measurements and/or determinations corresponding to values used to generate and/or modify an animal tissue model such as the animal tissue model generated via execution of process 200 and/or used in execution of process 300. In some embodiments, additional information may be received from one or more components of system 100. Exemplary additional information includes, but is not limited to, optical, physiological, and/or geometrical properties of the pregnant mammal's tissue and/or fetal tissue, fetal heart rate, maternal heart rate, phase differences between the fetal and maternal heart rates, equipment used to measure and/or determine the additional values, and/or information and/or measurements from one or more components of system 100.

In the case of a pregnant human, the measured output data received in step 422 may be one or more light transmission data sets that include an optical signal emanating from the pregnant human's abdomen responsively to one or more input optical signal(s) that is detected by a detector (e.g., detector 160) over an interval of time (e.g., 30-300 seconds) and converted into, for example, a digital and/or analog signa. The measured, or actual, output values may be measured in vivo fetal oximetry values corresponding, in time, to when the light transmission data sets were measured and/or detected. At times, measured in vivo fetal oximetry values may be within the range of, for example, of 10-70% of the fetal hemoglobin being oxygenated. In some cases, the set of set of measured, or actual, data received in step 422 may be converted into a format compatible with the predicted outputs of, for example, the first and/or second version of the simulated fetal oximetry model(s) so that a valid comparison between them may be made.

In step 424, instructions to adapt the first or second (when steps 416-420 are performed) version of the simulated fetal oximetry model for use in the generation of a first version of an in vivo fetal oximetry model may be received. The first version of the in vivo fetal oximetry model may be generated by training, tuning, and/or updating for example, the first/second version of the simulated fetal oximetry model using a plurality of measured in vivo light transmission data sets and corresponding measured in vivo fetal oximetry values.

Exemplary instructions received in step 424 include instructions to train, or update, only certain portions (e.g., layers, functions, networks, and/or sub-networks) of the first/second version of the simulated fetal oximetry model and fix, or hold constant, other portions of the fetal first/second version of the simulated fetal oximetry model as needed. Typically, the initial input layer or layers of the network would be fixed to preserve the features found in the simulations. Additionally, or alternatively, portions of the first/second version of the simulated fetal oximetry model that may remain fixed include portions of the first/second version of the simulated fetal oximetry model that are generally applicable to the in vivo fetal oximetry model such as, for example, layers pertaining to calibration factors, calibration curves, calibration formulas, maternal physiology and/or geometry, fetal physiology and/or geometry, and/or equipment parameters.

Optionally, in step 425, the measured in vivo light transmission data sets and corresponding output values (e.g., fetal oximetry values) may be divided into a measured training set and a measured testing set. In step 426, the in vivo light transmission data sets and corresponding output data (e.g., oximetry values) and/or the training set of in vivo light transmission data sets and corresponding output data (when step 424 is executed) may be input into an adapted (according to the instructions of step 424) version of the first/second version of the simulated fetal oximetry model so that one or more portions (e.g., layers, formulas, sub-formulas, and/or functions) of the first/second fetal oximetry model may be tuned or updated using the in vivo light transmission data and corresponding output values thereby generating an in vivo fetal oximetry model and, optionally, a third set of predicted output values may be generated by the in vivo fetal oximetry model.

In step 428, the third set of predicted output values may be compared with the corresponding measured output values to determine differences between them (step 428). Results of the comparison may then be evaluated (step 430) and used to update the in vivo fetal oximetry model (step 432). Execution of step 432 may also include storing the updated in vivo fetal oximetry model in a database such as the databases disclosed herein.

Optionally, when step 425 is performed, the testing set of measured light transmission data and corresponding output values may then be run through the in vivo fetal oximetry model to generate a fourth set of predicted output values (step 434). The fourth set of predicted output values may then be compared with the corresponding measured output values from the testing set of output values to determine differences between them (step 436). Results of the comparison may then be evaluated (step 438) and used to generate an updated in vivo fetal oximetry model to predict output values (step 440) using the machine learning architecture. The updated in vivo fetal oximetry model may also be stored in step 440. Then, the in vivo fetal oximetry model and/or an indication of the comparison(s), evaluation(s), and/or predicted output values may be provided to the user (step 442).

In some embodiments, process 400 and/or portions thereof may be repeated on a periodic, as-needed, and/or continuous basis to, for example, improve the accuracy of the predictions the in vivo fetal oximetry model yields, perform perturbation analysis, and/or perform sensitivity analysis. When step 434 is not performed, process 400 may end at step 432.

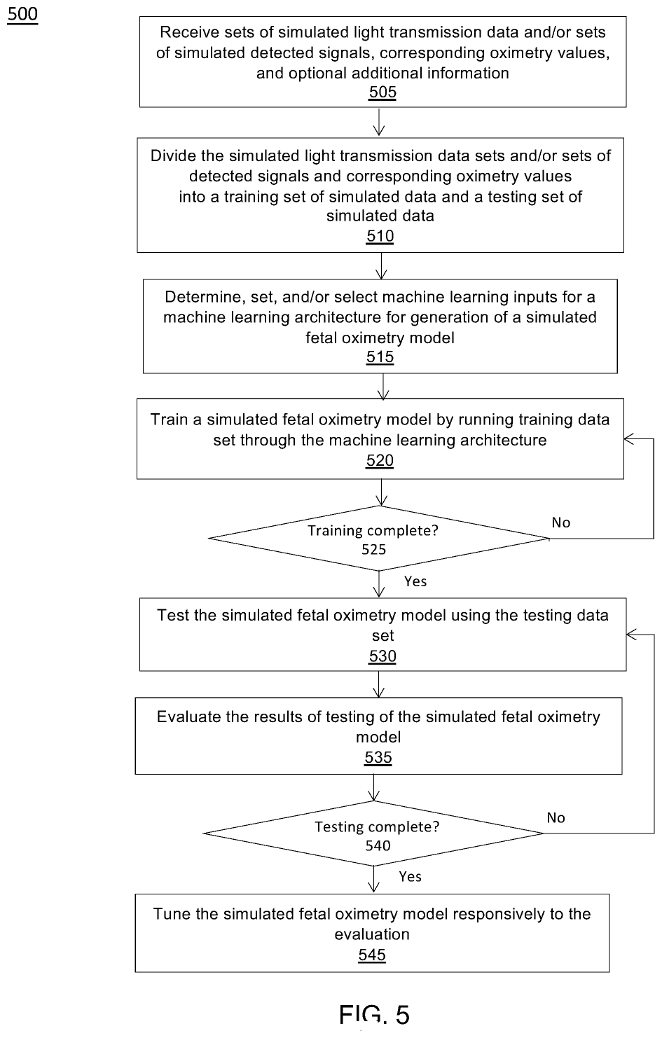

FIG. 5 is a flowchart illustrating an exemplary process 500 for the generation of a simulated fetal oximetry model and/or a tuned simulated fetal oximetry model. Process 500 may be performed by, for example, any of the systems or system components disclosed herein and may use data, determinations, and/or models generated and/or used by any of the processes disclosed herein and, in some cases, execution of process 500 may incorporate execution of one or more additional processes and/or process steps disclosed herein.

Initially, a plurality (e.g., 10,000-10 million) of sets of simulated light transmission data and corresponding oximetry values for each set of simulated light transmission data may be received by a processor or network of processors such as cloud computing platform 11 (step 505). The sets of simulated light transmission data may have been generated by, for example, execution of process 200 and/or 300. The oximetry values corresponding to each set of simulated light transmission data may have been generated via, for example, execution of process 200 and/or 300 and/or may be calculated as part of execution of step 505 using the simulated light transmission data. Optionally, in step 505, additional information regarding one or more of sets of simulated light transmission data and/or oximetry values may be received. The additional information may pertain to, for example, one or more of the inputs to the animal tissue model(s) used to generate sets of simulated light transmission data disclosed herein and may include, but are not limited to, fetal depth, source/detector separation distance, a thickness of maternal tissue, a type of maternal tissue, maternal and/or fetal skin color and/or melanin content, a thickness of fetal tissue, a type of fetal tissue, a type of light used, an intensity of light used, a light scattering property of layer of tissue in the model, a light absorption property of a layer of tissue in the model, a fetal age, a calibration formula, a calibration formula specific to a particular patient characteristic and/or patient, and/or calibration factor(s) associated with equipment used to obtain the simulated light transmission data, environmental conditions when the simulated light transmission data is collected.

Optionally, the plurality of sets of simulated light transmission data and corresponding fetal oximetry values may be divided into a training set of simulated data and a test set of simulated data (step 510). The plurality of sets of simulated light transmission data and corresponding fetal oximetry values may be divided along any appropriate ratio including, for example, 90:10 training/testing; 80:20 training/testing; or 70:30 training/testing. In some embodiments, execution of step 510 may be similar to execution of step 408.

In step 515, machine learning inputs for the generation of a simulated fetal oximetry model may be determined, set, and/or selected for input into a machine learning program and/or architecture such as herein described. In some embodiments, execution of step 515 may resemble execution of step 410. Then, in step 520, a simulated fetal oximetry model may be trained using all or most of the data (in all or most combinations) received in step 505 and/or the training set of data of step 510 when step 510 is executed. Step 520 may be executed via, for example, inputting the simulated light transmission data, simulated detected signals, corresponding oximetry values and/or addition information and/or a training set thereof (when step 510 is executed) into the machine learning architecture once it is set up with the machine learning inputs of step 515. The simulated fetal oximetry model may be configured to receive a plurality of sets of simulated light transmission data included in the training set of simulated data and determine an oximetry value for a fetus for each set of simulated light transmission data included in the training set of simulated data. This determined oximetry value may then be compared with the corresponding oximetry value received in step 505 to determine any differences therebetween. Results of this comparison may be used to iteratively update/train the simulated fetal oximetry model during execution of step 520. Training of the simulated fetal oximetry model may be complete (step 525) when, for example, a number or proportion (e.g., 60-99%) of the oximetry values calculated by the simulated fetal oximetry model using one or more simulated light transmission data sets received in step 505 are sufficiently close to (e.g., within a standard of deviation, within 0.5 standards of deviation, within 0.1 standards of deviation, and/or within 60-99% of the associated oximetry value) the oximetry values associated each of the respective simulated light transmission data sets. When the training of the simulated fetal oximetry model is not complete (step 525), step 520 may be repeated. When step 510 is not executed, process 500 may end following a determination that the training of the simulated fetal oximetry model in step 525 is complete.

In some embodiments, the simulated fetal oximetry model includes a plurality of layers, factors, calibrations, calibration formulas, and/or functions (referred to herein collectively as “layers”) that are used to calculate oximetry values using the simulated light transmission data. Layers may include functions that account for, and/or factor in, for example, fetal depth, source/detector separation distance, a thickness of maternal tissue, a type of maternal tissue, maternal and/or fetal skin color and/or melanin content, a thickness of fetal tissue, a type of fetal tissue, a wavelength of light used, an intensity of light used, a fetal age, calibration formulas, and/or calibration factor(s) associated with equipment that may be used in clinical applications to obtain in vivo measurements of light transmission data, environmental conditions that may be present during clinical applications when in vivo measurements of light transmission data is collected.

When the training of the simulated fetal oximetry model is complete (step 525), the simulated fetal oximetry model may be tested with the testing set of simulated data (step 530). In some embodiments, execution of step 530 may be similar to execution of step 416. Results of the testing of the simulated fetal oximetry model may then be evaluated (step 535) to, for example, determine how accurately the simulated fetal oximetry model calculated oximetry values. In some cases, the testing of step 530 may be iterative. When the testing of the simulated fetal oximetry model is complete (step 540), the simulated fetal oximetry model may be tuned responsively to one or more results of the testing and/or evaluation of the tests (step 545) thereby generating a tuned simulated fetal oximetry model and process 500 may end. When the testing of the simulated fetal oximetry model is not complete (step 540), process 500 may proceed to step 530.

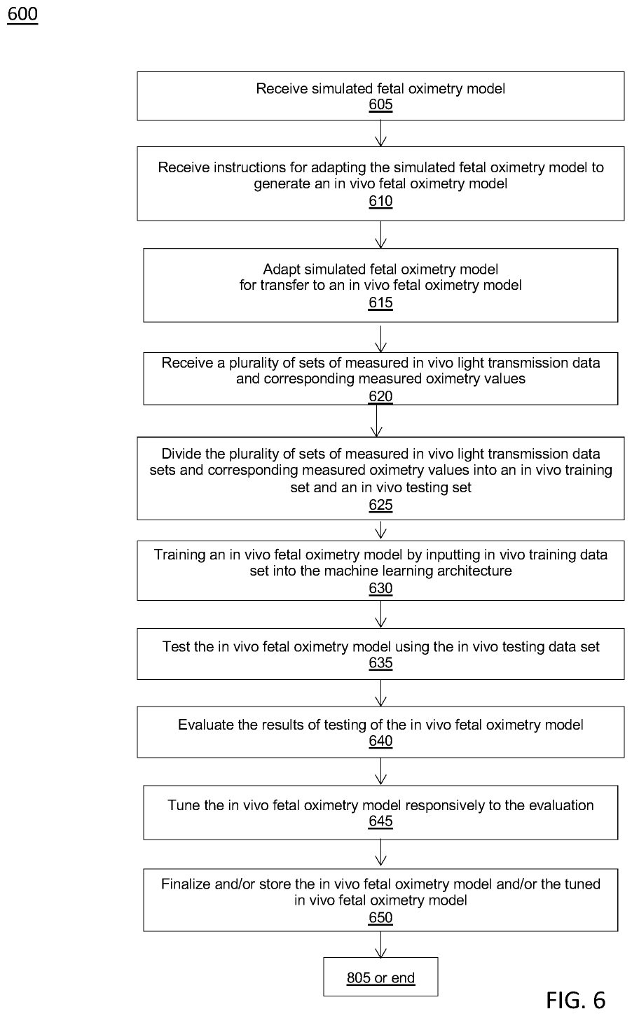

FIG. 6 is a flowchart illustrating an exemplary process 600 for the generation of an in vivo fetal oximetry model and/or a tuned in vivo fetal oximetry model. Process 600 may be performed by, for example, any of the systems or system components disclosed herein and may use data, determinations, and/or models generated and/or used by any of the processes disclosed herein. In some embodiments, process 600 may be performed subsequently to performance of process 500 and, on occasion, may be executed by the same systems and/or processors as process 500 and, in some cases, execution of process 600 may incorporate execution of one or more additional processes and/or process steps disclosed herein.

In step 605, a tuned simulated fetal oximetry model, such as the tuned simulated fetal oximetry model generated by process 500, may be received by, for example, a processor or network of processors such as cloud computing platform 11. Additionally, or alternatively, when, for example, steps 530-545 of process 500 are executed, a tuned simulated fetal oximetry model may be received in step 605. For ease of discussion, the following discussion of process 600 will use the phrase “simulated fetal oximetry model” to refer to both the simulated fetal oximetry model of, for example, step 525 of process 500 and the tuned simulated fetal oximetry model of, for example, step 545 of process 500.

Instructions to adapt the simulated fetal oximetry model for transfer to an in vivo fetal oximetry model may then be received (step 610). In some cases, the instructions to adapt the simulated fetal oximetry model for transfer to an in vivo fetal oximetry model may include instructions to fix one or more layers, or functions, of the simulated fetal oximetry model that may be generally applicable to the in vivo fetal oximetry model. Exemplary layers and/or functions of the tuned simulated fetal oximetry model that may be fixed include, but are not limited to, how one or more of a source/detector distance, a wavelength of light incident on the modeled pregnant mammal's abdomen, a fetal depth, maternal skin color, fetal skin color, maternal tissue composition, fetal tissue composition and/or a calibration factor impact (e.g., weights in the model), an oximetry calculation.

Then, the tuned simulated fetal oximetry model may be adapted for transfer to an in vivo fetal oximetry model responsively to the instructions (step 615). In some cases, the adapting of step 615 may include determining, setting, and/or selecting one or more machine learning inputs for a machine learning architecture for the generation of an in vivo fetal oximetry model. Additionally, or alternatively, the adapting of step 615 may include fixing one or more layers, or functions, of the tuned simulated fetal oximetry model so that it remains fixed during the in vivo fetal oximetry model training process (step 630, which is discussed below).

In step 620, a plurality (e.g., 1,000-10 million) of sets of in vivo light transmission data and corresponding fetal oximetry values for each set of in vivo light transmission may be received. The plurality of sets of in vivo light transmission data may be received from, for example, a fetal oximetry probe like fetal oximetry probe 115 and/or fetal oximetry probe system 117 and the corresponding fetal oximetry values may be calculated using, for example, Equations 1 and 2 as discussed herein.

Optionally, in step 625, the plurality of sets of in vivo light transmission data and corresponding fetal oximetry values may then be divided into a training set of in vivo data and a test set of in vivo data. The plurality of sets of in vivo light transmission data and corresponding fetal oximetry values may be divided along any appropriate ratio including, for example, 90:10 training/testing; 80:20 training/testing; or 70:30 training/testing. In some embodiments, execution of step 625 may have one or more similarities with execution of step 408 and/or 510.

In step 630, an in vivo fetal oximetry model may be trained using the training set of in vivo data and the adapted simulated fetal oximetry model of step 615. Step 630 may be executed via, for example, inputting the training set of in vivo data into the machine learning architecture once it is set up with the adapted simulated fetal oximetry model of step 615. In some embodiments, the in vivo fetal oximetry model may be configured to receive a plurality of sets of in vivo light transmission data included in the plurality of sets of measured in vivo data and/or training set of in vivo data and determine an oximetry value of a fetus for each respective set of in vivo light transmission data. This determined oximetry value may then be compared with the oximetry value associated with the in vivo light transmission data to determine any differences therebetween. These differences may be used to, for example, iteratively update/train the in vivo fetal oximetry model to, for example, improve accuracy and/or processing times during execution of step 630. Training of the in vivo fetal oximetry model may be complete when, for example, a number or proportion (e.g., 60-99%) of the oximetry values calculated by the in vivo fetal oximetry model using one or more in vivo light transmission data sets received in step 620 are sufficiently close to (e.g., within a standard of deviation, within 0.5 standards of deviation, within 0.1 standards of deviation, and/or within 60-99% of the associated oximetry value) to the oximetry values associated with each of the respective in vivo transmitted light data sets. When the training of the in vivo fetal oximetry model is not complete, step 630 may be repeated and/or may continue to be executed.

In some embodiments, the in vivo fetal oximetry model includes a plurality of layers, factors, calibrations, and/or functions (referred to herein collectively as “layers”) that are used to calculate oximetry values using the in vivo light transmission data. Exemplary layers include functions that factor in, account for, and/or are associated with one or more of inputs to an animal model as disclosed herein (see e.g., step 210 of process 200) and may include, but are not limited to, fetal depth, source/detector separation distance, a thickness of maternal tissue, a type of maternal tissue, maternal and/or fetal skin color and/or melanin content, a thickness of fetal tissue, a type of fetal tissue, a type of light used, an intensity of light used, a fetal age, calibration factor(s) associated with equipment used to obtain the in vivo light transmission data, and/or environmental conditions when the in vivo light transmission data is collected.

When the training of the in vivo fetal oximetry model is complete (step 630), process 600 may optionally proceed to step 650. Alternatively, and optionally, when the training of the in vivo fetal oximetry model is complete, the in vivo fetal oximetry model may be tested with the testing set of in vivo data (step 635). Optionally, results of the testing of the in vivo fetal oximetry model may then be evaluated (step 640) to, for example, determine how accurate the in vivo fetal oximetry model calculated oximetry values are. In some cases, the testing of step 635 may be iterative. When the testing of the in vivo fetal oximetry model is complete, the in vivo fetal oximetry model may be tuned and/or updated responsively to one or more results of the testing and/or evaluation of the tests (step 645) thereby generating a tuned in vivo fetal oximetry model. The tuned in vivo fetal oximetry model may then be finalized and/or stored (step 650) and process 600 may end and/or proceed to step 805 of process 800 as discussed below.

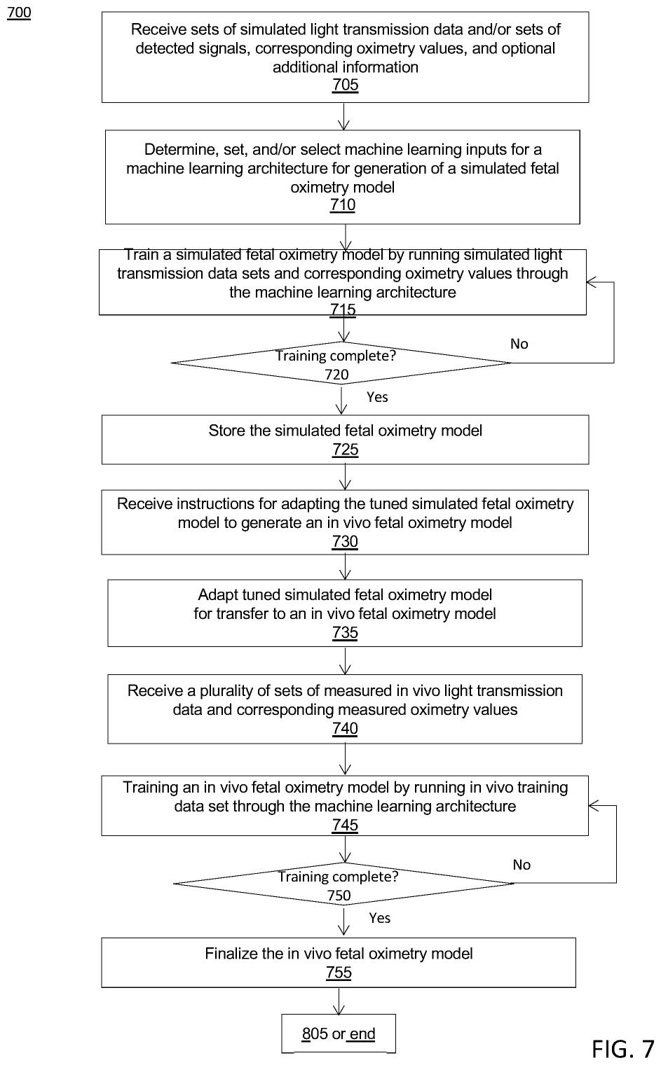

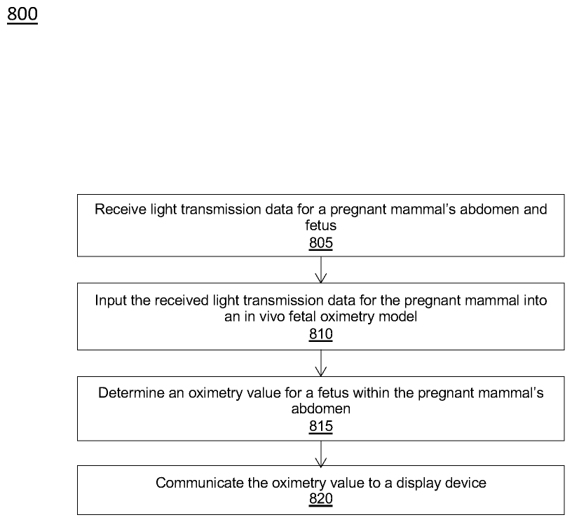

FIG. 7 is a flowchart illustrating another exemplary process 700 for the generation of an in vivo fetal oximetry model. Process 700 may be performed by, for example, any of the systems or system components disclosed herein and may use data, determinations, and/or models generated and/or used by any of the processes disclosed herein and, in some cases, execution of process 700 may incorporate execution of one or more additional processes and/or process steps disclosed herein.

Initially, a plurality (e.g., 100,000-10 million) of sets of simulated light transmission data and corresponding oximetry values for each set of simulated light transmission data may be received by a processor or network of processors such as cloud computing platform 11 (step 705). Each set of the simulated light transmission data may have been generated by simulating a transmission of light of one more wavelengths and/or intensities through a model of animal tissue that may have been generated and/or received via, for example, execution of process 200 and/or 300. The simulated light transmission data sets may resemble those received in, for example, step 404. In some embodiments, the oximetry values corresponding to each set of simulated light transmission data may have been generated via, for example, execution of process 200 and/or 300 and/or may be calculated as part of execution of step 705 using the simulated light transmission data. On some occasions, execution of step 705 may resemble execution of step 505.

In step 710, machine learning inputs for the generation of a simulated fetal oximetry model may be determined, set, and/or selected for input into a machine learning program and/or architecture such as TensorFlow. In some embodiments, execution of step 710 may resemble execution of step 410 and/or 515. Then, in step 715, a simulated fetal oximetry model may be trained using the simulated light transmission data sets and corresponding oximetry values. Step 715 may be executed via, for example, inputting the simulated light transmission data and corresponding oximetry values into the machine learning architecture once it is set up with the machine learning inputs of step 710. At times, execution of step 715 may resemble execution of step 520.

The simulated fetal oximetry model may be trained and/or configured to receive a plurality of sets of simulated light transmission data and determine an oximetry value for a fetus that may be associated with each set of simulated light transmission data. This determined oximetry value may then be compared with the oximetry value associated with respective sets of simulated light transmission data received in step 705 to determine any differences therebetween. Results of this comparison may be used to iteratively update/train the simulated fetal oximetry model during execution of step 715. Training of the simulated fetal oximetry model may be complete (step 720) when, for example, a number or proportion (e.g., 60-99%) of the oximetry values calculated by the simulated fetal oximetry model using one or more simulated light transmission data sets received in step 705 are sufficiently close to (e.g., within a standard of deviation, within 0.5 standards of deviation, within 0.1 standards of deviation, and/or within 60-99% of the associated oximetry value) of the oximetry values associated each of the respective simulated light transmission data sets. When the training of the simulated fetal oximetry model is not complete (step 720), step 715 may be iteratively repeated. In some embodiments, execution of step 720 may resemble execution of step 525.

In some embodiments, the simulated fetal oximetry model includes a plurality of layers, factors, calibrations, and/or functions (referred to herein collectively as “layers”) that are used to calculate oximetry values using the simulated light transmission data. Layers may include, for example, functions that account for and/or factor in, for example, one or more of the inputs of step 215 and/or fetal depth, source/detector separation distance, a thickness of maternal tissue, a type of maternal tissue, maternal and/or fetal skin color and/or melanin content, a thickness of fetal tissue, a type of fetal tissue, a type of light used, an intensity of light used, a fetal age, and/or calibration factor(s) associated with equipment that may be used in clinical applications to obtain in vivo measurements of light transmission data, environmental conditions that may be present during clinical applications when in vivo measurements of light transmission data is collected. When the training of the simulated fetal oximetry model is complete (step 720), it may be stored in a database like database 15 and/or 170 (step 725).

In step 730, instructions to adapt the simulated fetal oximetry model for transfer to an in vivo fetal oximetry model may then be received. In some cases, the instructions to adapt the simulated fetal oximetry model for transfer to an in vivo fetal oximetry model may include instructions to fix one or more layers, or functions, of the simulated fetal oximetry model that may be generally applicable to the in vivo fetal oximetry model so that these fixed layers/functions do not change during the training process. Exemplary layers and/or functions of the simulated fetal oximetry model that may be fixed include, but are not limited to, how one or more of a source/detector distance, a wavelength of light, a fetal depth, maternal skin color, fetal skin color, maternal tissue composition, fetal tissue composition and/or a calibration factor impact (e.g., weights in the model), an oximetry calculation. In some embodiments, execution of step 730 may resemble execution of step 424 and/or 610.

Then, the simulated fetal oximetry model may be adapted for transfer to an in vivo fetal oximetry model responsively to the instructions (step 735). In some cases, the adapting of step 735 may include determining, setting, and/or selecting one or more machine learning inputs for a machine learning architecture for the generation of an in vivo fetal oximetry model. Additionally, or alternatively, the adapting of step 735 may include fixing one or more layers, or functions, of the simulated fetal oximetry model so that it remains fixed during the in vivo fetal oximetry model training process (step 745, which is discussed below).

In step 740, a plurality (e.g., 500-10 million) of sets of in vivo light transmission data and corresponding fetal oximetry values for each set of in vivo light transmission may be received. The plurality of sets of in vivo light transmission data may be received from, for example, a fetal oximetry probe like fetal oximetry probe 115 and/or fetal oximetry probe system 117 and the corresponding fetal oximetry values may be calculated using, for example, Equations 1 and 2 as discussed herein. Then, in step 745, an in vivo fetal oximetry model may be generated and/or trained using the in vivo data and the adapted simulated fetal oximetry model of step 735. Step 745 may be executed via, for example, inputting a plurality of sets of in vivo data into the machine learning architecture once it is set up with the adapted simulated fetal oximetry model of step 735. The in vivo fetal oximetry model may be configured to receive a plurality of sets of in vivo light transmission data and determine an oximetry value of a fetus for each set of in vivo light transmission data included in the training set of in vivo data. This determined oximetry value may then be compared with the oximetry value associated with a respective set of in vivo light transmission data that may be received in step 740 to determine any differences therebetween. These differences may be used to, for example, iteratively update/train the in vivo fetal oximetry model during execution of step 745. Training of the in vivo fetal oximetry model may be complete (step 750) when, for example, a number or proportion (e.g., 60-99%) of the oximetry values calculated by the in vivo fetal oximetry model using one or more in vivo light transmission data sets received in step 740 are sufficiently close to (e.g., within a standard of deviation, within 0.5 standards of deviation, within 0.1 standards of deviation, and/or within 60-99% of the associated oximetry value) to the oximetry values associated with each of the respective in vivo transmitted light data sets.

In some embodiments, the in vivo fetal oximetry model includes a plurality of layers, factors, calibrations, and/or functions (referred to herein collectively as “layers”) that are used to calculate oximetry values using the in vivo light transmission data. The layers of the in vivo fetal oximetry model may correspond to one or more of the layers of the simulated fetal oximetry model. When the training of the in vivo fetal oximetry model is not complete, (step 745) may be repeated and/or may continue to be iteratively executed. When the training of the in vivo fetal oximetry model is complete (step 745), the vivo fetal oximetry model may then be finalized and stored (step 755) and process 700 may end or proceed to step 805 of process 800 discussed below.

FIG. 8 is a flowchart illustrating an exemplary process 800 for the determination of a fetal oximetry value for a fetus using an in vivo fetal oximetry model that may be generated via, for example, execution of process 600 and/or 700. Process 800 may be performed by, for example, any of the systems or system components disclosed herein and, in some cases, execution of process 800 may incorporate execution of one or more additional processes and/or process steps disclosed herein.

Initially, in step 805, light transmission data for a pregnant mammal's abdomen and fetus may be received from, for example, a photodetector like detector 160 and/or a probe like fetal oximetry probe 115 and/or fetal oximetry probe system 117. The received light transmission data may correspond to light from a light source (e.g., light source 105) that is incident on a pregnant mammal's abdomen and, in some instances, a fetus within the pregnant mammal's abdomen and emanates from the pregnant mammal's abdomen via, for example, backscattering from and/or transmission through abdominal/fetal tissue and is detected by a detector like detector 160. The light transmission data received in step 805 may then be put into, and/or processed using, a in vivo fetal oximetry model, such as the finalized in vivo fetal oximetry model of step 650 of process 600 and/or the finalized in vivo fetal oximetry model of step 755 of process 700 (step 810). In some embodiments, the light transmission data received in step 805 may be pre-processed prior to execution of step 810. The pre-processing may include, for example, filtering with, for example, a Kalman or bandpass filter, application of a noise reduction process or algorithm, removal of a portion of the light transmission data that is incident only the pregnant mammal (i.e., not incident on the fetus), and/or isolation of a portion of the light transmission data corresponding to light incident on the fetus from the light transmission data received in step 805. On some occasions, removal of a portion of the light transmission data that is incident only the pregnant mammal (i.e., not incident on the fetus), and/or isolation of a portion of the light transmission data that corresponds to light incident on the fetus from the received light transmission data may be accomplished by, for example, receiving a maternal heartrate signal, using the maternal heart rate signal to identify the portion of the light transmission data contributed by the pregnant mammal and then subtracting the portion of the light transmission data contributed by the pregnant mammal from the light transmission data. Additionally, or alternatively, isolation of the fetal portion of the light transmission data may be accomplished by, for example, receiving a fetal heartrate signal, using the fetal heart rate signal to identify the portion of the light transmission data contributed by the fetus and then subtracting the remainder of light transmission data and/or amplifying the portion of the light transmission data contributed by the fetus. Additionally, or alternatively, isolation of the fetal portion of the light transmission data may include determining a fetal position and/or fetal depth, determining how long it would take (e.g., time of flight) a photon and/or optical signal incident on the maternal abdomen to reach the fetus and be detected by the detector, and then using this time of flight for photons/the detected optical signal to filter out photons/portions of the detected optical signal that were not in flight long enough to have reached the fetus.

In step 815, an oximetry value for the fetus within the pregnant mammal's abdomen may be determined and/or output by the in vivo fetal oximetry model. The oximetry value may be, for example, a fetal hemoglobin oxygen saturation level, a fetal tissue oxygen saturation level, an indication of fetal hypoxia, an indication of fetal hypoxemia, and/or an alert condition indicating that a fetal oximetry value indicates the fetus may be in distress. The oximetry value may then be communicated to a display device like display device 14 and/or 155 for display to a user such as a clinician and/or the pregnant mammal via, for example, one or more of the interfaces and/or graphic user interfaces (GUIs) disclosed herein.

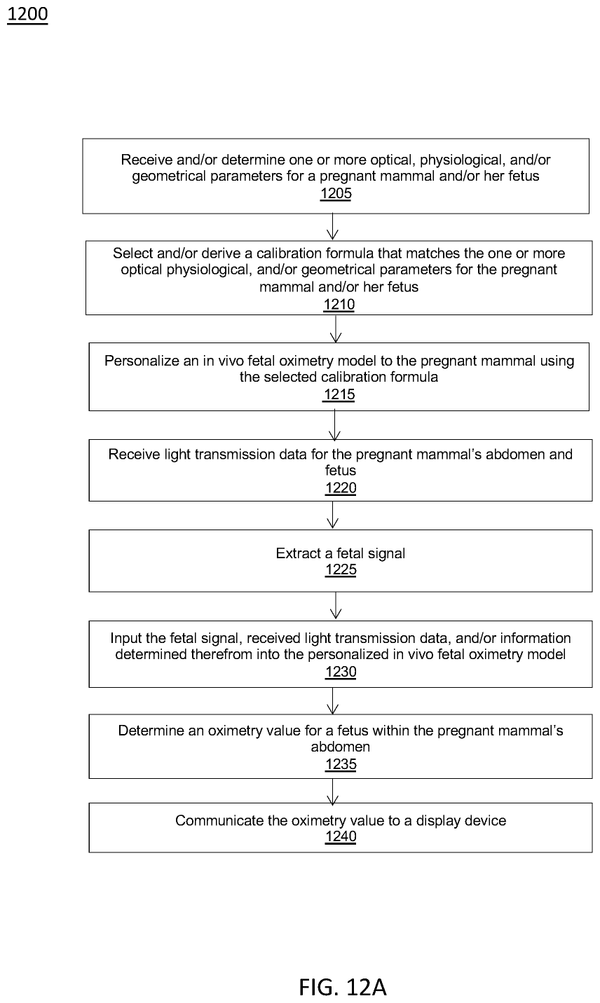

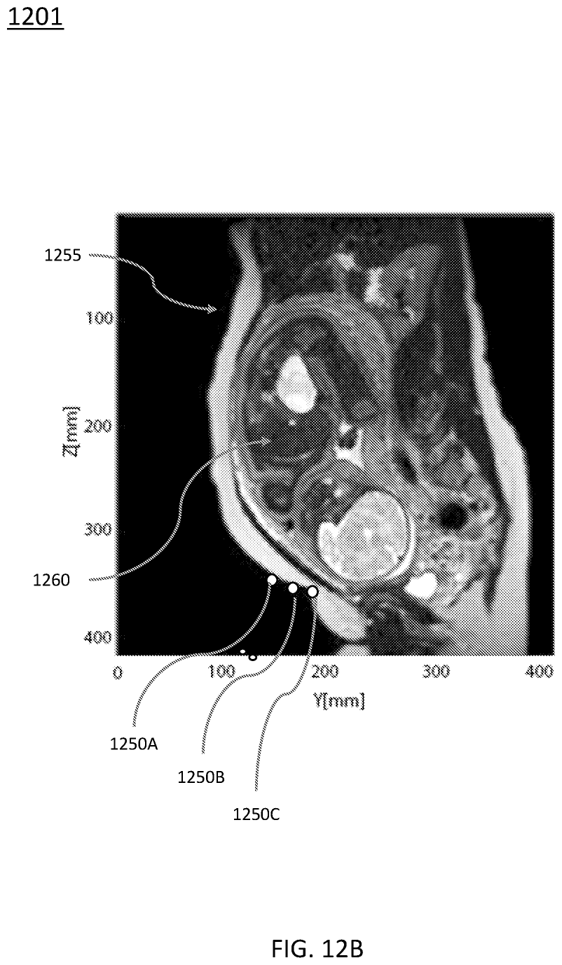

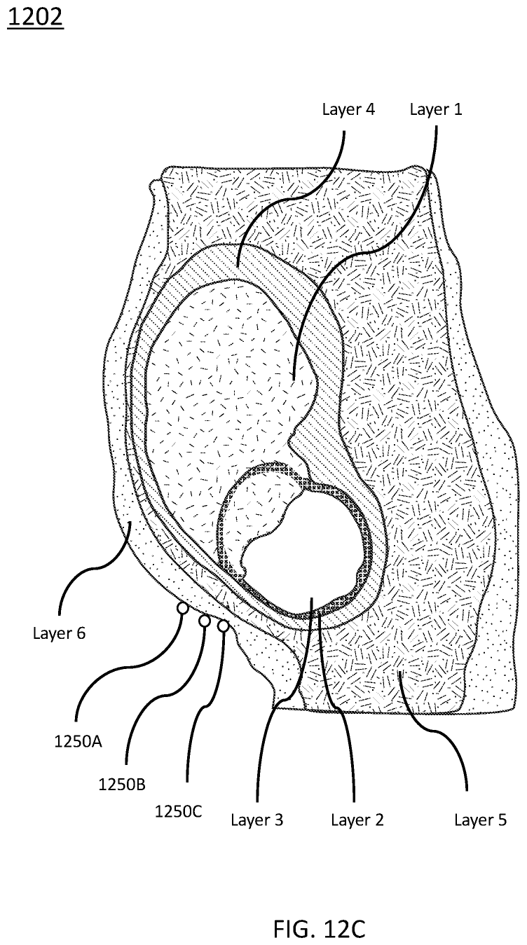

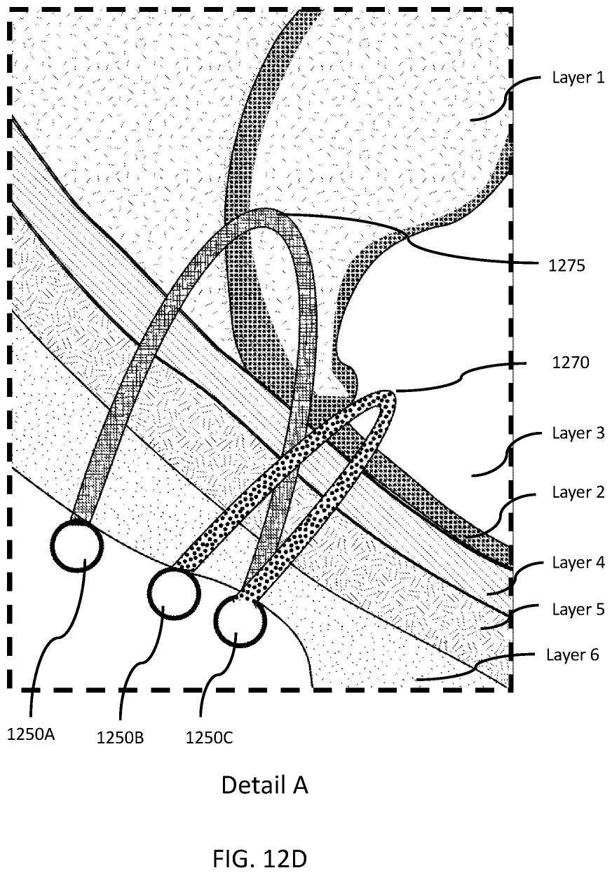

FIG. 12 provides a flowchart of an exemplary process 1200 for using an in vivo fetal oximetry model to determine a fetal oxygenation value. Process 1200 may be executed by, for example, any of the systems or system components disclosed herein and, in some cases, execution of process 1200 may incorporate execution of one or more additional processes and/or process steps disclosed herein.

Initially, one or more optical, physiological, and/or geometrical properties of a pregnant mammal and/or her fetus may be received and/or determined (step 1205). Additionally, or alternatively, and/or one or more optical and/or operational properties of equipment used to determine a fetal oximetry value may be received and/or determined in step 1205. Exemplary optical features include, but are not limited to, light scattering and/or light absorption coefficients for the maternal and/or fetal tissue that may be known and/or determined via, for example, execution of a frequency domain (e.g., FFT) analysis of an optical signal corresponding to light that has traveled through the maternal abdomen and/or analysis of time of flight for photons detected upon emission from a pregnant mammal's abdomen. Exemplary physiological features include, but are not limited to, maternal oximetry information, maternal and/or fetal skin color, and maternal body mass index. Exemplary geometrical features include, but are not limited to, fetal depth, a thickness of one or more layers of maternal tissue the light passes through, a part of the fetus (e.g., head, back, face, etc.) light is incident upon, and a fetal position.

When one or more optical and/or operational properties of equipment used to determine a fetal oximetry value are received in step 1205, these properties may provide, for example, wavelengths of light emitted, lag time, whether the detector provides a digital or analog output, whether or not the optical signals emitted and/or detected by the equipment are time stamped and, if so, how they are time stamped, and/or distortions introduced into emitted and/or detected signals by the equipment. The equipment used to determine a fetal oximetry value may include, for example, fetal oximetry probe 115 and/or fetal oximetry probe system 117.

On some occasions, fetal depth may be deduced using, for example, relative distances between a light source and one or more detectors that detects light transmission data that includes light incident upon the fetus. For example, in an array of four detectors placed in a linear configuration at a distance of 1 cm, 2 cm, 3 cm, and 4 cm from the light source if the second detector (at a distance of 2 cm from the light source) detects light transmission data that includes light incident upon the fetus it may be deduced that the fetus is relatively shallow (i.e., fetal depth is relatively small) and using the geometry of the source/detector distance between the light source and the second detector, a fetal depth may be deduced. Likewise, if only the fourth detector (at a distance of 4 cm from the light source) detects light transmission data that includes light incident upon the fetus, it may be deduced that the fetus is relatively deep (i.e., fetal depth is relatively large) and using the geometry of the source/detector distance between the light source and the fourth detector, a fetal depth may be deduced.

On some occasions, light absorption by the pregnant mammal and/or fetus may be responsive to skin pigmentation and/or a level of melanin in the skin of the pregnant mammal and/or fetus. At times, skin color may be received in step 1205 to assist with the determination of light absorption characteristics of the pregnant mammal and/or fetus. Skin color of the pregnant mammal may be quantified using, for example, the Fitzpatrick scale.

Additionally, or alternatively, light scattering properties of the pregnant mammal may be a function of tissue layer composition (e.g., skin, adipose, muscle) and relative thicknesses of the tissue layers for her abdomen. This information may be provided by, for example, a two-dimensional and/or three-dimensional image generated via, for example, an imaging technique such as ultrasound and/or MRI scan such as such as MRI image 1201 of FIG. 12B, which is a cross section of a pregnant woman's abdomen 1255 and a fetus 1260 contained therein that shows dimensions (in mm) in the Z and Y dimensions. In particular, image 1201 shows a pregnant mammal's abdomen 1255 and fetus 1260, layers and regions of maternal and fetal tissue, an optional first position marker 1250A, an optional second position marker 1250B, and an optional third position marker 1250C. First, second, and/or third position markers 1250A, 1250B, and/or 1250C may serve to, for example, mark a position of, for example, imaging equipment (e.g., ultrasound wand) and/or oximetry equipment such as one or more light source(s) like light source 105, detector(s) such as detector 160, fetal oximetry probes like fetal oximetry probe 115, and/or fetal oximetry probe systems like fetal oximetry probe system 117. In some instances, first, second, and/or third position markers 1250A, 1250B, and/or 1250C may be provided as coordinates (e.g., X-, Y, and/or Z-coordinates) along with the image in addition to, or instead of, being visually represented on the image. In some embodiments, first, second, and/or third position markers 1250A, 1250B, and/or 1250C may correspond to marks made on the pregnant mammal's abdomen that may be used to position an imaging device (e.g., ultrasound wand), oximetry equipment such as one or more light source(s) like light source 105, detector(s) such as detector 160, fetal oximetry probes like fetal oximetry probe 115, and/or fetal oximetry probe systems like fetal oximetry probe system 117 in a known and/or consistent location by, for example, placing the imaging device, light source, and/or detector on top of, and/or at a fixed position relative to, the mark and/or determining a position of the imaging device, light source, and/or detector relative to first, second, and/or third position markers 1250A, 1250B, and/or 1250C via, for example, manually measuring a distance and/or angle between them and/or using an automated measuring device (e.g., a IR measurement device). The marks of first, second, and/or third position markers 1250A, 1250B, and/or 1250C may be made by, for example, manually marking the skin of the pregnant mammal with, for example, a permanent marker and/or placing a sticker or lead on the pregnant mammal's abdomen.

Once image like image 1201 is received, execution of step 1210 may include processing and/or analyzing the image to determine one more features, such as one or more of a geometrical, anatomical, physiological property tissue type, position, size, shape and/or of the fetus and/or pregnant mammal. This processing may include, for example, digitization of image 1201, applying one or more noise reduction processes to image 1201, applying one or more contrast amplification and/or image resolution improvement processes to image 1201, and/or analysis of image 1201 using object and/or image recognition software to, for example, identify different objects (e.g., uterine wall, fetal head, fetal back, etc.), regions, and/or types (e.g., muscle, adipose tissue, and/or bone) of tissue for the pregnant mammal and/or fetus. An exemplary output of this processing is provided by FIG. 12C, which shows a digitized rendering 1202 of image 1201 following processing and/or analysis. FIG. 12D provides a detailed view of a portion of rendering 1202. Rendering 1202 provides a key that is color/grey scale coded to show different types of tissue, wherein layer 1 is fetal tissue, layer 2 is fetal skull tissue, layer 3 is fetal brain tissue, layer 4 is amniotic fluid, layer 5 is uterine wall tissue, and layer 6 is maternal fat, or adipose, tissue. It will be noted that not all 10 layers are shown in rendering 1202 but, these layers may be included in other exemplary images like image 1201. Once the layers of image 1201 are digitized and/or rendered (as shown in, for example, FIG. 12C), one or more optical, physiological, and/or geometrical parameters for pregnant mammal 1255 and/or fetus 1260 may be determined and these determined optical, physiological, and/or geometrical parameters may be used to define, select, and/or build a calibration, equation, formula and/or factor (or a portion thereof) that may, for example, be incorporated into an in vivo fetal oximetry model as, for example, explained herein.

In some cases, the optical, geometrical, and/or physiological parameters for a pregnant mammal and her fetus that are received in step 1205 include a location and/or position on the maternal abdomen of one or more light sources, such as light source 105, detectors such as detector 160, imaging devices, and/or fetal oximetry probes like fetal oximetry probe 115 and/or fetal oximetry probe system 117. The location and/or position information may be absolute (e.g., a set of X-, Y-, and/or Z-coordinate) as may be determined by, for example, a global positioning system component positioned within the light source(s), detector(s), and/or fetal oximetry probe. Additionally, or alternatively, location and/or position information for a source, detector, imaging device, fetal oximetry probe, and/or fetal oximetry probe system may be relative position and/or location information, wherein a position of a light source, detector, imaging device, fetal oximetry probe, and/or fetal oximetry probe system may be relative to, for example, one or more location markers like location markers 1250A, 1250B, and/or 1250C, and/or an anatomical feature of the pregnant mammal's abdomen such as the navel and/or a bone (e.g., the pelvic bone). In some embodiments, an orientation of a source, detector, imaging device, and/or fetal oximetry probe may also be received in step 1205 from, for example, an accelerometer, present within the respective light source, detector, imaging device, fetal oximetry probe, and/or fetal oximetry probe system. Orientation information may be used to, for example, determine an angle of incident light as it is projected into the maternal abdomen and/or an angle of light that is incident upon a detector like detector 160. Additionally, or alternatively, orientation information may be used to determine a pathway an optical signal has likely traveled through the maternal abdomen and, in some cases, fetus to eventually be detected by detector.

In step 1210, a calibration formula, or a set of calibration formulas, that match the one or more optical, physiological, and/or geometrical parameters of a pregnant mammal and/or her fetus received and/or determined in step 1205 may be determined, derived, and/or selected. In some cases, the calibration formulas may be similar to the calibration formulas that define the best fit lines shown in graphs 201 and/or 202. Additionally, or alternatively, the calibration formulas may correspond to scattering and/or absorption characteristics for the maternal tissue positioned between the fetus and the light source and/or detector. These scattering and/or absorption characteristics may be based upon, for example, tissue type, tissue thickness, fetal depth, maternal skin color, and/or fetal skin color.

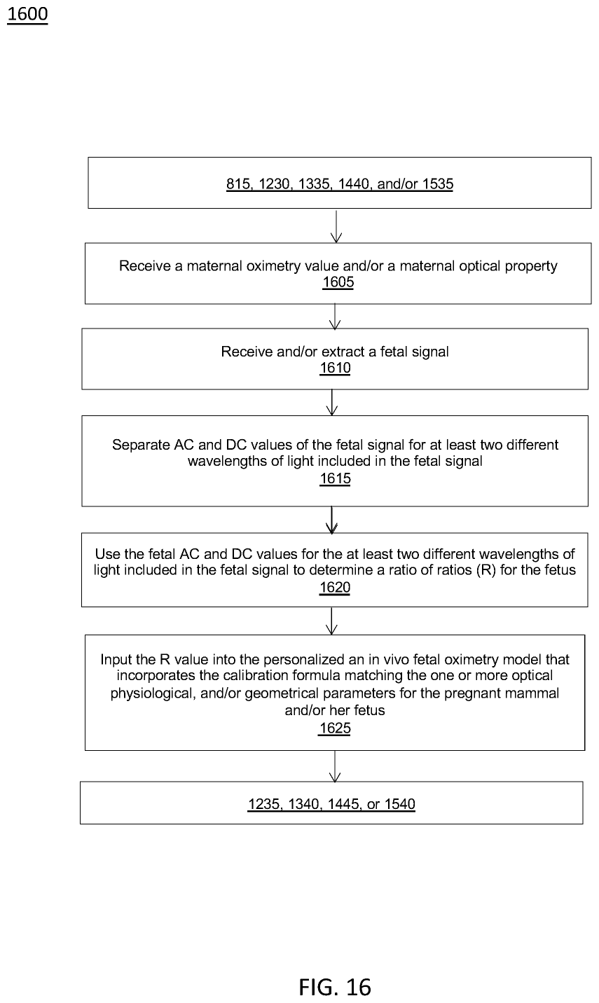

At times, execution of step 1210 includes querying a database of various calibration formulas and/or calibration formulas such as database 15 and/or 170 or a portion thereof for a calibration formula that matches some or all of the parameters of step 1205. The selected calibration formula may be used to personalize an in vivo fetal oximetry model to the pregnant mammal (step 1215). At times, execution of step 1215 may include adjusting one or more inputs and/or processes of the in vivo fetal oximetry model. Further details on how step 1215 may be performed are provided below with regard to process 1600 of FIG. 16.

Returning to the example of FIGS. 12B, 12C, and 12D, once the anatomical and/or geometrical features of fetus 1260 and maternal abdomen 1255 are processed, digitized, and/or rendered (as shown in FIGS. 12C and 12D), they may be analyzed to determine one or more anatomical (e.g., tissue type, tissue composition, etc.), geometrical (e.g., size, shape, thickness, etc.), and/or optical properties (e.g., scattering, absorption, optical density, and/or time of flight) thereof via, for example, execution of one or more processes disclosed herein. At times, one or more optical properties of pregnant mammal 1255 and/or fetus 1260 may be deduced and/or calculated using one or more anatomical and/or geometrical properties determined via, for example, generation and/or analysis of rendering 1202. For example, if it a thickness of one or more layers of different types of maternal issue that are in an optical path (e.g., in a path between a light source and a detector) is determined using, for example, the rendering and/or a process described herein, then an experimentally determined and/or known scattering and/or absorption coefficient for each of the tissue types may also be deduced and/or added to a calibration formula.

As noted herein, the physiological and/or geometrical properties of the maternal abdomen 1255 and/or fetus 1260 may be used to determine optical properties thereof. In the example of FIG. 12D, light traveling along a first optical path 1270 travels from a light source positioned at third location marker 1250C, through layers 10, 7, 6, 3, and 2, to a detector positioned at second location marker 1250B and light traveling along a second optical path 1275 travels from a light source positioned at third location marker 1250C, through layers 10, 7, 6, 2, and 1, to a detector positioned at first location marker 1250A. In some embodiments, a geometrical property (e.g., width) of each of the layers along first and/or second optical paths 1270 and 1275 and/or an optical property (e.g., scattering, absorption, and/or time of flight) may be used to, for example, define, calculate, and/or generate one or more calibration equations, formulas, and/or curves (or a portion thereof) as disclosed herein. In this way, a calibration equation, formula, and/or curve may be personalized to pregnant mammal 1255 and/or fetus 1260.

At times, processing of an image of a pregnant mammal, fetus, and/or a digitization thereof (e.g., rendering 1202) to define, calculate, and/or generate one or more calibration equations, formulas, and/or curves (or a portion thereof) may be executed using one or more optical analysis software programs such as Monte Carlo simulations and/or calculations using the NIRFAST platform. In some circumstances, this processing may include determining a calibration equation (step 1215) for a path of light that is incident on the pregnant mammal's abdomen at a particular location (e.g., a position corresponding to first location marker 1250A) and is detected by a detector positioned at a second particular location (e.g., a position corresponding to second location marker 1250B). The calibration equation may factor in optical properties such as scattering and/or absorption characteristics and/or coefficients for the different types/layers of tissue the light passes through, the width of the tissue the light passes through, and/or the fetal depth.

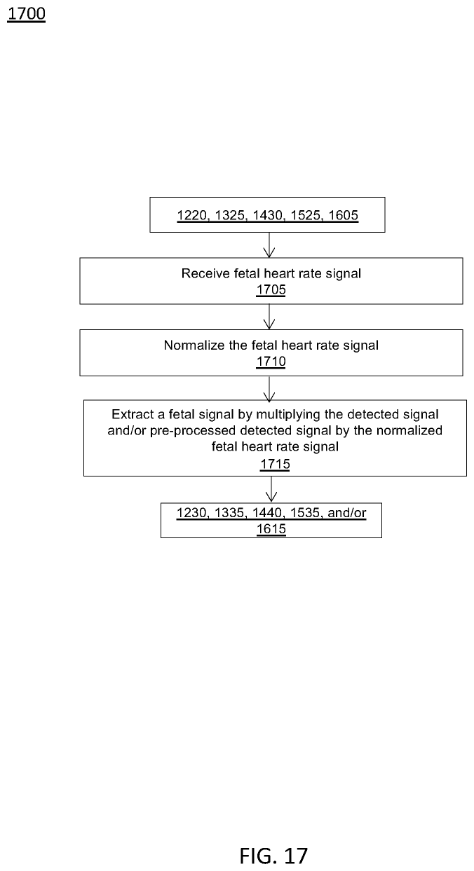

In step 1220, light transmission data (e.g., a detected signal that corresponds to an optical signal of two or more wavelengths) for light that has emanated from the pregnant mammal's abdomen and fetus may be received from, for example, a fetal oximetry probe like fetal oximetry probe 115 and/or fetal oximetry probe system 117. The light transmission data may be processed to isolate a fetal signal (step 1225) that corresponds to light that was incident upon the fetus. Further details regarding how step 1225 may be performed are provided below with regard to processes 1700 and/or 1800 of FIGS. 17 and/or 18, respectively.

The fetal signal, received light transmission data, and/or information determined therefrom may then be input into the personalized in vivo fetal oximetry model (step 1230), an oximetry value for the fetus may be determined (step 1235), and the oximetry value for the fetus may be communicated to a display device for observation by, for example, a clinician and/or the pregnant mammal (step 1240). Personalizing the in vivo fetal oximetry model may include, for example, adding, subtracting, and/or modifying one or more features, portions, and/or formulas of the in vivo fetal oximetry model to incorporate, or factor in, data relating to the pregnant mammal and/or fetus. This personalization of the in vivo fetal oximetry model enables more accurate calculation of a fetal oximetry value in step 1235 because, for example, the calculation incorporates features specific to the particular pregnant mammal and fetus being studied. Further details regarding how steps 1230 and 1235 may be performed are provided below with regard to process 1600 of FIG. 16.

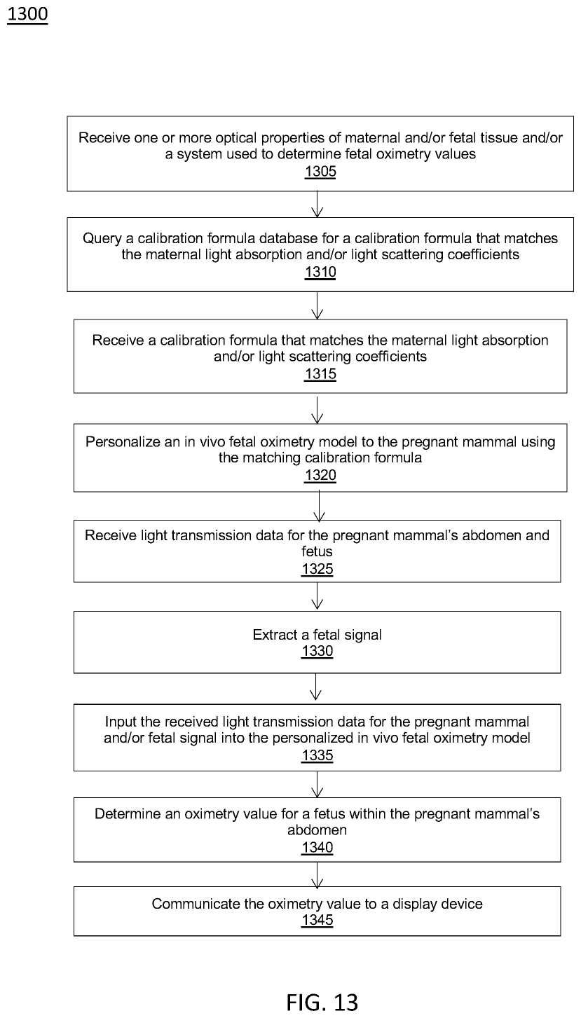

FIG. 13 provides a flowchart of an exemplary process 1300 for selecting a calibration formula for use with an in vivo fetal oximetry model and determining a fetal oxygenation value using the calibration formula and an in vivo fetal oximetry model. Process 1300 may be executed by, for example, any of the systems or system components disclosed herein and, in some cases, execution of process 1300 may incorporate execution of one or more additional processes and/or process steps disclosed herein.

Initially, one or more optical properties of a pregnant mammal and/or fetus and/or one or more optical and/or operational properties of equipment used to determine a fetal oximetry value may be received (step 1305). Exemplary optical properties include a time of flight for photons of an optical signal incident on the pregnant mammal's abdomen to be detected by a detect, a light scattering coefficient for the maternal and/or fetal tissue, and/or a light absorption coefficient for the maternal and/or fetal tissue. In some embodiments, the light scattering and/or light absorption coefficients for the maternal tissue may be determined via analysis (e.g., FFT), and/or analysis of time of flight for of an optical signal corresponding to light that only passes through maternal tissue as may be the case with, for example, a short-separation measurement. At times this optical signal and/or a digital signal corresponding to it may be received from first light/source detector system 107. In some embodiments, execution of step 1305 may resemble execution of step 1205.

When one or more optical and/or operational properties of equipment used to determine a fetal oximetry value are received in step 1305, these properties may provide, for example, wavelengths of light emitted, lag time, whether the detector provides a digital or analog output, whether or not the optical signals emitted and/or detected by the equipment are time stamped and, if so, how they are time stamped, and/or distortions introduced into emitted and/or detected signals by the equipment. The equipment used to determine a fetal oximetry value may include, for example, fetal oximetry probe 115 and/or fetal oximetry probe system 117.

In step 1310, a database of various calibration formulas (e.g., the calibration formulas of graphs 201 and 202) such as database 15 and/or 170 or a portion thereof, may be queried for a calibration formula that matches, or is associated with, one or more of the optical properties received in step 1305. In some cases, the query of step 1310 may specify that the returned calibration formula must match two or more optical properties (e.g., both the light scattering coefficient and the light absorption coefficient for the tissue of the pregnant mammal and/or fetus). Additionally, or alternatively, the query of step 1310 may request two or more calibration formulas to apply and/or input into an in vivo fetal oximetry model.

In step 1315, one or more calibration formula(s) that match and/or associated with the optical properties of the pregnant mammal, fetus, and/or equipment may be received and used to personalize an in vivo fetal oximetry model to the pregnant mammal (step 1320). The calibration formulas may, in some cases, be selected and/or configured to correct for signal distortions caused by, for example, the equipment, fetal tissue, and/or maternal tissue. At times, execution of step 1320 may include adjusting one or more inputs, subroutines, and/or processes of the in vivo fetal oximetry model to personalize it to the pregnant mammal's optical properties, the equipment being used to determine fetal oximetry values for the pregnant mammal, and/or environmental or other conditions (e.g., ambient light, background noise, etc.) that may be specific to a situation in which a fetal oximetry measurement is being taken and/or a fetal oximetry value is being determined. Further details on how step 1320 may be performed are provided below with regard to process 1600 of FIG. 16.

In step 1325, light transmission data (e.g., a detected signal that corresponds to an optical signal of two or more wavelengths) for light that has emanated from the pregnant mammal's abdomen and fetus may be received from, for example, a fetal oximetry probe like fetal oximetry probe 115 and/or fetal oximetry probe system 117 and/or second source/detector system 167. The light transmission data may be processed to isolate a fetal signal (step 1330) that corresponds to light that was incident upon the fetus. Further details regarding how step 1330 may be performed are provided below with regard to processes 1700 and/or 1800 of FIGS. 17 and/or 18, respectively.

The fetal signal, received light transmission data, and/or information determined therefrom may then be input into the personalized in vivo fetal oximetry model (step 1335), an oximetry value for the fetus may be determined (step 1340), and the oximetry value for the fetus may be communicated to a display device for observation by, for example, a clinician and/or the pregnant mammal (step 1345) via, for example, one or more of the interfaces and/or GUIs disclosed herein. Further details regarding how steps 1340 and 1345 may be performed are provided below with regard to process 1600 of FIG. 16.

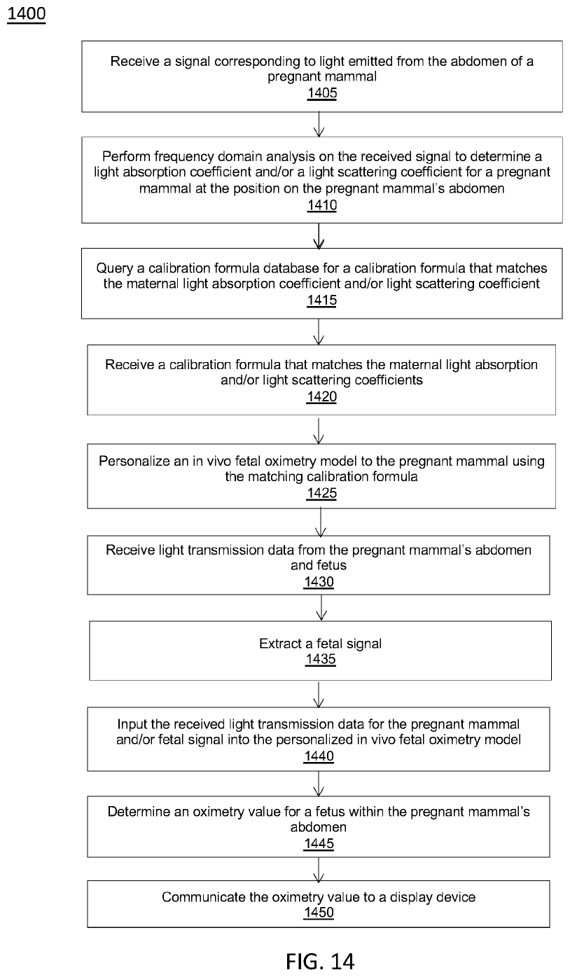

FIG. 14 provides a flowchart of an exemplary process 1400 for determining optical properties of maternal tissue, selecting a calibration formula for use with an in vivo fetal oximetry model responsively to the maternal optical properties, and determining a fetal oxygenation value using the calibration formula and an in vivo fetal oximetry model. Process 1400 may be executed by, for example, any of the systems or system components disclosed herein and, in some cases, execution of process 1400 may incorporate execution of one or more additional processes and/or process steps disclosed herein.

Initially, a signal corresponding to light emitted from the abdomen of a pregnant mammal (also referred to herein as light transmission data) may be received (step 1405). In some cases, the signal received in step 1405 may not include light that was incident on the fetus and may be, for example, a short separation signal that only penetrates maternal tissue. In step 1410, frequency domain (e.g., FFT) analysis and/or analysis of time of flight for photons detected upon emission from a pregnant mammal's abdomen may be performed on the received signal to determine light scattering and/or light absorption coefficients for the maternal tissue. In some embodiments, a position of a probe providing the signal received in step 1405 may also be received in step 1405 and this position may be used to, for example, determine the optical properties of the pregnant mammal at a position near and/or at a known distance from where the light transmission data corresponding to light that was incident on the fetus. In some embodiments, the position information received in step 1405 may resemble the position information received in step 1205 or the position information for first, second, and/or third position markers 1250A, 1250B, and/or 1250C, respectively, as discussed above with regard to FIGS. 12B-12D.

In step 1415, a database of various calibration formulas (e.g., such the calibration formulas of graph 201 and 202), such as database 15 and/or 170 or a portion thereof, may be queried for a calibration formula that matches light scattering and/or light absorption coefficients for the maternal tissue of step 1405. Often times, the query of step 1415 may specify that the returned calibration formula must match both the light scattering coefficient and the light absorption coefficient for the maternal tissue. In step 1420, a calibration formula that matches light scattering and/or light absorption coefficients for the maternal may be received and used to personalize an in vivo fetal oximetry model to the pregnant mammal (step 1425). At times, execution of step 1425 may include adjusting one or more inputs, subroutines, and/or processes of the in vivo fetal oximetry model to personalize it to the pregnant mammal's light scattering and/or light absorption coefficients. Further details on how step 1425 may be performed are provided below with regard to process 1600 of FIG. 16.

In step 1430, light transmission data (e.g., a detected signal that corresponds to an optical signal of two or more wavelengths) for light that has emanated from the pregnant mammal's abdomen and fetus may be received from, for example, a fetal oximetry probe like fetal oximetry probe 115 and/or fetal oximetry probe system 117 and/or second source/detector system 167. The light transmission data may be processed to isolate a fetal signal (step 1435) that corresponds to light that was incident upon the fetus. Further details regarding how step 1435 may be performed are provided below with regard to processes 1700 and/or 1800 of FIGS. 17 and/or 18, respectively.

The fetal signal, received light transmission data, and/or information determined therefrom may then be input into the personalized in vivo fetal oximetry model (step 1440), an oximetry value for the fetus may be determined using the personalized in vivo fetal oximetry model (step 1445), and the oximetry value for the fetus may be communicated to a display device for observation by, for example, a clinician and/or the pregnant mammal (step 1450) via, for example, one or more of the interfaces and/or GUIs disclosed herein. Further details regarding how steps 1445 and 1450 may be performed are provided below with regard to process 1600 of FIG. 16.

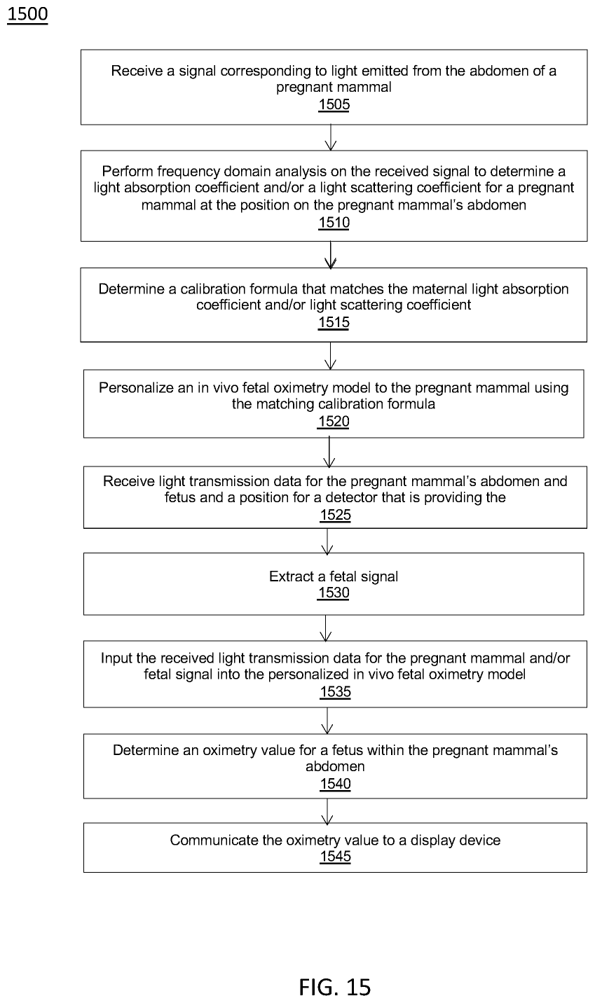

FIG. 15 provides a flowchart of another exemplary process 1500 for determining optical properties of maternal tissue, selecting a calibration formula for use with an in vivo fetal oximetry model responsively to the maternal optical properties, and determining a fetal oxygenation value using the calibration formula and an in vivo fetal oximetry model. Process 1500 may be executed by, for example, any of the systems or system components disclosed herein and, in some cases, execution of process 1500 may incorporate execution of one or more additional processes and/or process steps disclosed herein.

Initially, a signal corresponding to light emitted from the abdomen of a pregnant mammal (also referred to herein as light transmission data) may be received (step 1505). In some cases, the signal received in step 1505 may not include light that was incident on the fetus and may be, for example, a short separation signal that only penetrates maternal tissue. In step 1510, frequency domain (e.g., FFT) analysis and/or analysis of time of flight for photons detected upon emission from a pregnant mammal's abdomen may be performed on the received signal to determine light scattering and/or light absorption coefficients for the maternal tissue. In some embodiments, a position of a probe (e.g., fetal oximetry probe 115 and/or fetal oximetry probe system 117) providing the signal received in step 1505 and/or a component of the probe (e.g., light source 105 and/or detector 160) may also be received in step 1505 as, for example, described above with regard to FIGS. 12A-12D. In some embodiments, this position may be used to, for example, determine the optical properties of the pregnant mammal at a position near and/or at a known distance from where the light transmission data corresponding to light that was incident on the fetus.

In step 1515, a calibration formula (e.g., such the calibration formulas described herein and/or depicted in graph(s) 201 and 202) may be calculated or otherwise determined using, for example, the light scattering and/or light absorption coefficients, or other optical properties for the maternal tissue that may have been received in step 1505. The calibration formula determined in step 1515 may then be used to personalize an in vivo fetal oximetry model to the pregnant mammal (step 1520). At times, execution of step 1520 may include adjusting one or more inputs, subroutines, and/or processes of the in vivo fetal oximetry model to personalize it to the pregnant mammal's light scattering and/or light absorption coefficients. Further details on how step 1520 may be performed are provided below with regard to process 1600 of FIG. 16.

In step 1525, light transmission data (e.g., a detected signal that corresponds to an optical signal of two or more wavelengths) for light that has emanated from the pregnant mammal's abdomen and fetus may be received from, for example, a fetal oximetry probe like fetal oximetry probe 115, fetal oximetry probe system 117, and/or second source/detector system 167. The light transmission data may be processed to isolate a fetal signal (step 1530) that corresponds to light that was incident upon the fetus. Further details regarding how step 1530 may be performed are provided below with regard to processes 1700 and/or 1800 of FIGS. 17 and/or 18, respectively.

The fetal signal, received light transmission data, and/or information determined therefrom may then be input into the personalized in vivo fetal oximetry model (step 1535), an oximetry value for the fetus may be determined (step 1540), and the oximetry value for the fetus may be communicated to a display device for observation by, for example, a clinician and/or the pregnant mammal (step 1545). Further details regarding how steps 1540 and 1545 may be performed are provided below with regard to process 1600 of FIG. 16.

FIG. 16 provides a flowchart of an exemplary process 1600 for determining a fetal oximetry value of using a calibration formula and an in vivo fetal oximetry model. Process 1600 may be executed by, for example, any of the systems or system components disclosed herein following, for example, execution of step(s) 815, 1230, 1335, 1440, or 1535, of process 1200, 1300, 1400, or 1500 respectively.

Initially, in step 1605, a maternal oximetry value (e.g., a PPG DC value or signal) and/or a maternal optical property (e.g., absorption, scattering, and/or a maternal short-separation signal) may be received. Optionally, in step 1605, the maternal DC value may be received from, for example, a pulse oximeter like pulse oximetry probe 130 and/or NIRS adult hemoglobin probe 125. A fetal signal may then be received and/or generated (step 1610) via, for example, isolating a fetal contribution to a set of light transmission data (e.g., the light transmission data received in step 810, 1220, 1325, 1430, and/or 1525) using, for example, one or more of the processes described herein. In many cases, the fetal signal includes a PPG AC and a PPG DC value for multiple wavelengths of light that are incident upon a pregnant mammal's abdomen. The fetal AC and DC values may then be extracted from the fetal signal (step 1615). On some occasions, execution of step 1615 may include subtracting all AC signals and the maternal DC signal from light transmission data corresponding to light emanating from the pregnant mammal's abdomen such as the light transmission data received in step(s) 805, 1220, 1325, 1430, and 1525; with the remainder of the DC portion of the light transmission data being a fetal DC signal for one or more wavelengths of light. Additionally, or alternatively, separating the AC values of the fetal signal may include subtracting all DC signals and the maternal AC signal from the light transmission data; with the remainder being the AC signals, or values, contributed by light incident upon the fetus (i.e., fetal AC signals) for one or more wavelengths of light. Then, in step 1620, a ratio of ratios (R) may be calculated for the fetus according to, for example, Equations 3 and/or 4, as disclosed herein.

The R value of step 1620 may then be, for example, used as a calibration factor, used to generate a calibration formula, used to select a calibration formula, and/or used to generate a personalized in vivo fetal oximetry model as part of, for example, execution of steps 1235, 1340, 1445, or 1540 of process 1200, 1300, 1400, or 1500, respectively, to determine an oximetry value for the fetus.

FIG. 17 provides a flowchart of an exemplary process for generating a fetal signal. Process 1700 may be executed by, for example, any of the systems or system components disclosed herein and, in some cases, may executed as a subroutine of one or more of the processes disclosed herein.

Following execution of step 1220, 1325, 1430, 1525, and/or 1605, a fetal heart rate signal may be received from, for example, a Doppler/ultrasound probe like Doppler/ultrasound probe 135 and/or an ECG like ECG 175 (step 1705). Optionally, the fetal heart rate signal may be normalized (step 1710) and/or synchronized, in time, with, for example, the light transmission data and/or fetal signal. In some embodiments, the normalization of step 1710 may include adjusting values of one or more measurements and/or components of the detected signal (e.g., intensity magnitudes for different wavelengths of light) to be on a similar, or common, scale so that the different values may be more easily evaluated/analyzed. The fetal heart rate signal of step 1705 or the normalized fetal heart rate signal of step 1710 may then be multiplied by the light transmission data received in, for example, step 1220, 1325, 1430, and 1525 of process 1200, 1300, 1400, or 1500, respectively, to generate the fetal signal (step 1715).

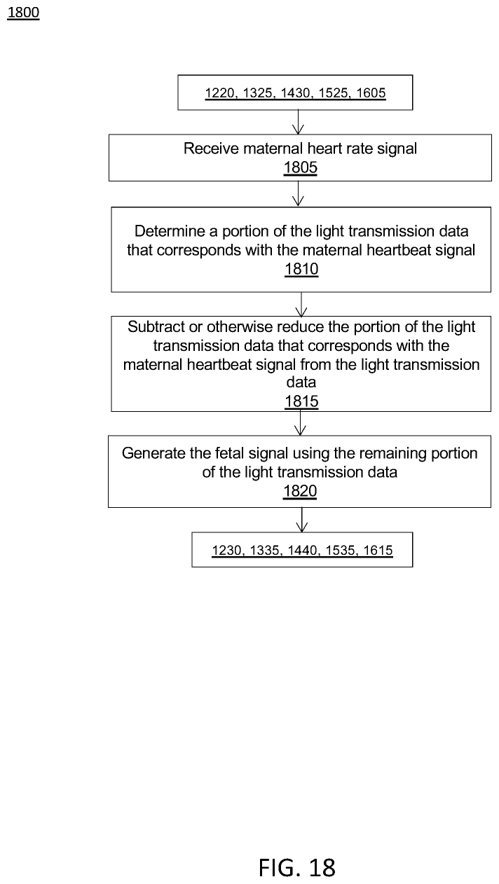

FIG. 18 provides a flowchart of another exemplary process for generating a fetal signal. Process 1800 may be executed by, for example, any of the systems or system components disclosed herein.

In step 1805, a maternal heart rate signal may be received, and a portion of the light transmission data received in, for example, step 1220, 1325, 1430, 1525, and/or 1605 of process 1200, 1300, 1400, 1500, and/or 1600, respectively may be analyzed to determine a portion of thereof that corresponds to the heartbeat signal of the pregnant mammal (step 1810). At times, this analysis may include synchronizing the maternal heart rate signal and the light transmission data in time and then comparing the light transmission data with the heartbeat signal of the pregnant mammal. Then, the portion of the light transmission data that corresponds to the heartbeat signal of the pregnant mammal may be subtracted from and/or regressed out of the multiplied signal via, for example, a linear regression expression, or otherwise reduced, or removed, from the light transmission data (step 1815), thereby generating a fetal signal using the remaining portion of the light transmission data (step 1820). Following execution of step 1820, step 1220, 1325, 1430, 1525, and 1615 of process 1200, 1300, 1400, 1500 and/or 1600, respectively, may be executed.

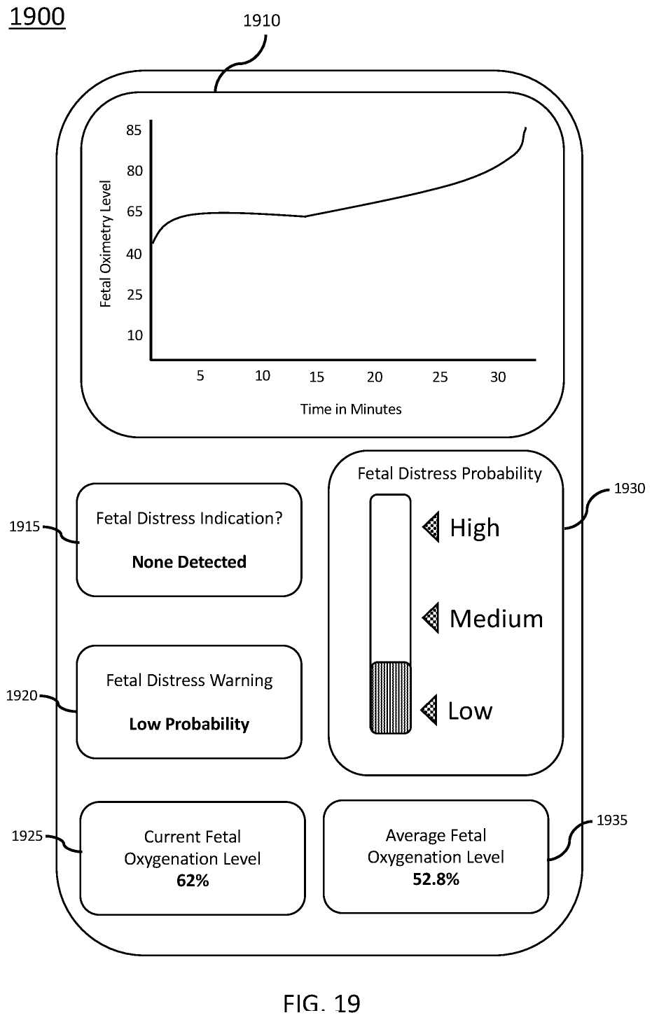

FIG. 19 is a screen shot of an exemplary user interface, or graphic user interface (GUI) 1900 that may be configured to display a result (e.g., a fetal oximetry value and/or indication of fetal distress) of executing one or more processes disclosed herein via, for example, one or more windows, icons, graphics, and/or text provided thereon. GUI 1900 may be displayed on, for example, display device 155, display device 14, and/or computer 13 responsively to instructions from, for example, computer 13 and/or cloud computing platform 11, and/or a component thereof (e.g., a processor, ASIC, and/or FPGA).

GUI 1900 includes a graph window 1910 that plots a plurality, or series of fetal oximetry level determinations, measurements, readings, and/or calculations taken over a period of time, in this instance 30 minutes. GUI 1900 further includes a fetal distress indication window 1915, a fetal distress warning window 1920, a current fetal oxygenation level window 1925, a fetal distress probability graphic window 1930, and an average fetal oxygenation level window 1935.

Fetal distress indication window 1915 may, for example, provide one or more messages regarding whether or not an indication of fetal distress has been detected such as “none detected” (as shown in FIG. 19). Other exemplary messages for fetal distress indication window 1915 include “potential distress detected” and “distress detected.” Additionally, or alternatively, fetal distress indication window 1915 may provide a message indicating an error condition and/or that additional information, or measurements, may be needed to assess fetal distress. Fetal distress warning window 1920 may, for example, an indication of a probability that the fetus is in distress such as “low probability” (as shown in FIG. 19). Other exemplary messages fetal distress warning window 1920 include, but are not limited to, a message indicating a probability level via words (e.g., high, medium, low) and/or numbers (e.g., 1-3; 1-5; 1-10; 1-100, etc.). Current fetal oxygenation level window 1925 may numerically display a value (in this case 62%) indicating a fetal oximetry level on any appropriate scale ((e.g., 1-3; 1-5; 1-10; 1-100, etc.)

Fetal distress, probability graphic window 1930 may graphically display a level of probably fetal distress as, for example, a bar graph (as shown in FIG. 19), a pie chart, a graph, and/or as a set of differently colored light that are coded to represent different levels of fetal distress probability (e.g., red indicate high distress probability and green indicates low distress probability). Average fetal oxygenation level window 1935 may display an average and/or a time-weighted average fetal oximetry value and/or level (in this case 52.8%) that may indicate an average fetal oximetry value over an interval of time (e.g., 5, 10, 15, 30, and/or 60 minutes).

Although FIG. 19 is in black and white, this need not always be the case as one or more windows of interface 1900 may provide information in the form of colors (e.g., red indicate high distress probability and green indicates low distress probability) that, in some cases, may indicate a, for example, a change in the fetus' oximetry level and/or a change in the fetus' distress probability. Additionally, or alternatively, a display device providing GUI 1900 may provide audible alerts and/or messages indicating, for example, a change in the fetus' oximetry level, a change in the fetus' distress probability, and the like.

It will be understood by those of skill in the art that GUI 1900 may not, in all circumstances, include each and every window shown in FIG. 19. In some embodiments, a GUI configured to display a result (of executing one or more processes disclosed herein may only include graph window 1910 and fetal distress, probability graphic window 1930. Alternatively, a GUI configured to display a result (of executing one or more processes disclosed herein may include fetal distress indication window 1915 and numerical value for a current fetal oxygenation level window 1925.

In some embodiments disclosed herein, one or more trained models, simulated fetal oximetry models, in vivo fetal oximetry models, calibration equations and/or correction factors may be used in combination to, for example, determine oximetry values for a patient, a fetus, and/or a pregnant mammal.

1. A method comprising:
receiving, by a processor, an image of a pregnant mammal's abdomen;
analyzing, by the processor, the image to determine one or more characteristics of the pregnant mammal's abdomen;
receiving, by a processor, light transmission data, the light transmission data corresponding to an optical signal that is detected by a photodetector and converted into the light transmission data, the optical signal being a composite of light that is incident on the pregnant mammal's abdomen and a fetus disposed within the pregnant mammal's abdomen;
inputting, by the processor, the light transmission data into a personalized in vivo fetal oximetry model, the personalized in vivo fetal oximetry model incorporating a result of the analysis of the image; and
receiving, by the processor, an output from the personalized in vivo fetal oximetry model, the output including an indication of an oximetry value for the fetus.

2. (canceled)

3. The method of claim 1, wherein a result of the analysis is at least one of an optical characteristic of the pregnant mammal and/or fetus, a geometrical characteristic of the pregnant mammal and/or fetus, and a physiological characteristic of the pregnant mammal and/or fetus.

4. (canceled)

5. The method of claim 1, further comprising:
determining a calibration formula for the pregnant mammal responsively to a result of the analysis of the image, wherein the calibration formula is incorporated into the personalized in vivo fetal oximetry model.

6. The method of claim 1, further comprising:
determining, by the processor, an indication of fetal distress using the fetal oximetry value; and
providing, by the processor, the indication of fetus distress a to a display device.

7. (canceled)

8. (canceled)

9. The method of claim 1, further comprising:
determining, by the processor, a calibration equation for the pregnant mammal responsively to the analysis, wherein the calibration equation is incorporated into the personalized in vivo fetal oximetry model.

10. The method of claim 1, further comprising:
querying, by the processor, a database of calibration equations for a calibration equation for the pregnant mammal, the query requesting a calibration equation that is responsive to the analysis;
receiving, by the processor, a calibration equation from the database; and
incorporating, by the processor, the received calibration equation into the personalized in vivo fetal oximetry model.

11. A method comprising:
determining information regarding a blood oxygen value of a fetus, the determining comprising:
obtaining at least one signal indicating light detected from a pregnant mammal's abdomen and/or a fetus disposed in the pregnant mammal's abdomen following application of light to the pregnant mammal's abdomen; and
analyzing the at least one signal using a trained model personalized to the pregnant mammal to determine blood oxygen information used to determine the information regarding a blood oxygen value of the fetus.

12. The method of claim 11, wherein the trained model is a first model, the method further comprising:
analyzing the at least one signal using a second trained model to determine the blood oxygen information.

13. The method of claim 11, wherein the trained model is a first model, the method further comprising:
analyzing the at least one signal using a second trained model personalized to the pregnant mammal to determine the blood oxygen information.

14. The method of claim 11, wherein the trained model is personalized to the pregnant mammal using at least one of an optical, geometric, and physiological characteristic of the pregnant mammal's abdomen.

15. (canceled)

16. The method of claim 11, wherein the trained model is personalized to the pregnant mammal using at least one of a light scattering coefficient and a light absorption coefficient specific to the pregnant mammal.

17. The method of claim 11, wherein the trained model is personalized to the pregnant mammal using at least one of a skin color of the pregnant mammal's abdomen and a blood oxveen level of the Dregnant mammal.

18. The method of claim 11, wherein the trained model is personalized to the pregnant mammal using an image of the pregnant mammal's abdomen.

19. (canceled)

20. The method of claim 11, wherein the trained model is personalized to the pregnant mammal using a calibration formula that is responsive to one or more characteristics of the pregnant mammal.

21. The method of claim 11, wherein the trained model is trained using both results of simulations of light applications to pregnant mammal abdomens and results of light applications to pregnant mammal abdomens.

22. (canceled)

23. (canceled)

24. The method of claim 11, further comprising:
determining whether the fetus has fetal hypoxia or fetal hypoxemia using the blood oxygen value; and
providing an indication of a determination that the fetus has fetal hypoxia or fetal hypoxemia to a display device.

25-34. (canceled)

35. A computer-implemented method comprising:
receiving an image of a pregnant mammal's abdomen;
analyzing the image to determine one or more characteristics of the pregnant mammal's abdomen;
receiving optical data regarding the pregnant mammal's abdomen and physiological data for the pregnant mammal; and
generating a calibration equation for the pregnant mammal responsively to a result of the analysis of the image, the received optical data, and the received physiological data.

36. The computer-implemented method of claim 35, further comprising:
receiving light transmission data, the light transmission data corresponding to an optical signal that is incident on the pregnant mammal's abdomen and a fetus disposed within the pregnant mammal's abdomen and is detected by a photodetector and converted into the light transmission data; and
determining an oximetry value for the fetus by applying the calibration equation to the light transmission data.

37. The computer-implemented method of claim 35, further comprising:
receiving at least one of optical data for the fetus and physiological data for the fetus, wherein the calibration equation is further responsive to the received at least one of optical data for the fetus and physiological data for the fetus.

38. The computer-implemented method of claim 37, wherein the at least one of the physiological and optical data for the fetus is received from at least one of a frequency domain near infrared spectroscopy system, a continuous wave near infrared spectroscopy system, and a time of flight near infrared spectroscopy system.

39. The computer-implemented method of claim 35, wherein the light transmission data is received from at least one of a frequency domain near infrared spectroscopy system, a continuous wave near infrared spectroscopy system, and a time of flight near infrared spectroscopy system.

40. The computer-implemented method of claim 35, wherein the optical signal is a first optical signal, the photodetector is a first photodetector of a first spectroscopy system and the light transmission data is a first set of light transmission data, the method further comprising:
receiving a second set of light transmission data, the second set of light transmission data corresponding a second optical signal that is incident on the pregnant mammal's abdomen and a fetus disposed within the pregnant mammal's abdomen and is detected by a photodetector of a second spectroscopy system and converted into the second set of light transmission data; and
determining an oximetry value for the fetus by applying the calibration equation to the second set of light transmission data.

41. The computer-implemented method of claim 40, wherein the second spectroscopy system is a continuous wave near infrared spectroscopy system.

42. The computer-implemented method of claim 35, wherein at least one of the physiological and optical data for the pregnant mammal are received from a frequency domain near infrared spectroscopy system.

43. The computer-implemented method of claim 35, wherein at least one of the physiological and optical data for the pregnant mammal are received from a time of flight near infrared spectroscopy system.

44. The computer-implemented method of claim 35, wherein the physiological data for the pregnant mammal includes an oxygen saturation level for the pregnant mammal.

45-51. (canceled)
